(19) United States
(12) Patent Application Publication
(10) Pub. No.: US 2014/0005069 A1
(43) Pub. Date: Jan. 2, 2014

(54) GLYCOPROFILING WITH MULTIPLEXED SUSPENSION ARRAYS

(71) Applicants: Loretta Yang, San Diego, CA (US); Robert J. Woods, Athends, GA (US)

(72) Inventors: Loretta Yang, San Diego, CA (US); Robert J. Woods, Athends, GA (US)

(73) Assignees: GLYCOSENSORS AND DIAGNOSTICS, LLC., Athens, GA (US); UNIVERSITY OF GEORGIA RESEARCH FOUNDATION, INC., Athens, GA (US)

(21) Appl. No.: 14/001,702

(22) PCT Filed: Mar. 1, 2012
(86) PCT No.: PCT/US12/27211

§ 371 Date: Sep. 18, 2013

Related U.S. Application Data
(60) Provisional application No. 61/447,925, filed on Mar. 1, 2011.

(51) Int. Cl. G01N 33/53; G01N 33/66

(52) U.S. Cl. CPC G01N 33/5308; G01N 33/66
USPC 506/9; 506/18; 506/16

(57) ABSTRACT

The present invention is includes compositions and methods directed to the multiplexed analysis of carbohydrates and carbohydrate containing compounds. The compositions and methods utilize suspension array technology (SAT) and an array of different carbohydrate binding molecules, each carbohydrate binding molecules with a known carbohydrate binding specificity, to obtain a glycoprofile of the carbohydrate structure(s) in a sample. Each carbohydrate binding molecule of a given specificity is linked to the external surface of a population of individually addressable particles.

### CONTINUING APPLICATION DATA

This application claims the benefit of U.S. Provisional Application Ser. No. 61/447,925, filed Mar. 1, 2011, which is incorporated by reference herein.

### BACKGROUND

Unlike protein sequences, which are encoded by the organism's genetic material, the subsequent attachment of complex carbohydrates (glycans) in eukaryotes is controlled by enzymes that either trim or extend the glycan core. A single protein frequently exhibits multiple versions of the glycan, depending on the age or location of the protein. Variations in the glycosylation pattern (glycoprofile) can also result from a range of diseases that introduce mutations into gene sequences, or that alter regulatory control pathways. Aberrant protein glycosylation is therefore a hallmark of several disease states, including diabetes (Coppo and Amore, 2004, Kidney International; 65(5):1544-1547), IgA nephropathy (Amore and Coppo, 2000, Nephron; 86(3):255-259), and various cancers (Krengel et al., 2004, J Biol Chem; 279(7):5597-5603). Because of their exposure on cell surfaces, the glycan chains frequently also serve as receptors for viral and bacterial pathogens (Lim et al., 2008, J Proteome Res; 7(3):1251-63). The ability to characterize glycoprofiles is therefore relevant to disease marker discovery, the development of therapeutics, the study of infectious diseases, and glycobiology research in general. Moreover, the Food and Drug Administration (FDA) requires that the glycoprofiles of all therapeutic glycoproteins fall within accepted limits (Comer et al., 2001, Anal Biochem; 293:169-177). The biologics market is estimated at $100B-$117B annually and is the most rapidly growing sector of the pharmaceutical industry (Abbott et al., 2008, J Proteome Res; 7(4):1470-80; Kaneda et al., 2002, J Biol Chem; 277(19):16928-16935).

Currently, rapid and affordable tools for determining or monitoring protein glycosylation patterns do not exist. Instead, glycoprofiling typically employs techniques, such as mass spectrometry (MS) (Bechtel et al., 1990, J Biol Chem; 265(4):2028-2037), which are dependent on costly instrumentation and highly trained personnel. These technologies are also poorly suited for real-time monitoring of glycoprofiles, as for example during the expression of therapeutic proteins. There is a need for rapid, simple, reliable, and affordable tools for determining or monitoring protein glycosylation patterns.

### SUMMARY OF THE INVENTION

The present invention includes a composition having a plurality of individually addressable particles, each individually addressable particle having an external surface and having linked to said external surface a separate carbohydrate binding molecule.

In some embodiments of the composition, the carbohydrate binding molecules are independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains, and aptamers. In some embodiments, the LECTENZ molecule is derived from an enzyme selected from the group consisting of a glycosidase enzyme, a glycosyltransferase enzyme, polysaccharide lyase enzyme, sulfatase enzyme, a sulfotransferase enzyme, a ligase enzyme, an amidase enzyme, and an epimerase enzyme. In some embodiments, the LECTENZ molecule is derived from PNGaseF or O-GlcNAcase.

In some embodiments of the composition, individually addressable particles include beads or nanoparticles.

In some embodiments of the composition, each individually addressable particle is separately labeled with a detectable label. In some embodiments, the detectable label is an optically encoded fluorescent dye.

In some embodiments, the composition is formulated for flow cytometry analysis.

In some embodiments, the composition is formulated for image based analysis.

In some embodiments, the composition is formulated for research, industrial, medical, or veterinary use.

The present invention includes kits including a composition as described herein, packaging materials and instructions for use.

The present invention includes kits having one or more compositions, each composition having individually addressable particles; each individually addressable particle having an external surface and having linked to said external surface a separate carbohydrate binding molecule; and each individually addressable particle separately labeled with a detectable label.

In some embodiments, a kit further includes a secondary detection reagent for detectably labeling an analyte.

In some embodiments, a kit further includes positive and/or negative analyte controls.

In some embodiments, a kit further includes instructions for use.

In some embodiments, a kit is formulated for research, industrial, medical, or veterinary use.

In some embodiments, a kit is formulated for flow cytometry analysis.

In some embodiments, a kit is formulated for image based analysis.

In some embodiments, a kit further includes a software component to assist in the calculation of relative glycan proportions in a sample.

The present invention includes a multiplex detection method for detecting a carbohydrate or a carbohydrate containing compound in a sample, the method including contacting the sample with a solution having a plurality of individually addressable particles, each individually addressable particle having an external surface and having linked to said external surface a separate carbohydrate binding molecule; and detecting the binding of the carbohydrate or carbohydrate containing compound to one more individually addressable particles; wherein the carbohydrate or carbohydrate containing compound bound to one more individually addressable particles remains in suspension.

In some embodiments of the method, detecting a carbohydrate or carbohydrate containing compound includes detecting the structure of the carbohydrate.

In some embodiments of the method, each separate carbohydrate binding molecules is independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains (such as cholera toxin B, other toxins, and hemagglutinin), aptamers including protein, RNA or other small molecule aptamers, and any other molecule that naturally binds or is engineered to bind a carbohydrate.

In some embodiments of the method, the individually addressable particles include beads and/or nanoparticles.

In some embodiments of the method, each individually addressable particle is separately labeled with a detectable label. In some embodiments, the detectable label is an optically encoded fluorescent dye.

In some embodiments of the method, detection is by flow cytometry analysis.

In some embodiments of the method, detection is by image based analysis.

In some embodiments of the method, at least one of the detected carbohydrates or carbohydrate containing compounds is detectable labeled. In some embodiments, the method further includes co-detecting the detectably labeled individually addressable particle and the detectably labeled carbohydrates or carbohydrate containing compounds.

In some embodiments of the method, the carbohydrate includes at least one monosaccharide.

In some embodiments of the method, the carbohydrate includes a polymer including at least two monosaccharides, and wherein detecting the structure of the carbohydrate includes detecting at least one feature selected from the group consisting of constituent monomer, functional group, linkage position, linkage stereochemistry, presence or absence of branching, branch position.

In some embodiments of the method, the carbohydrate or carbohydrate containing compound is selected from the group consisting of a monosacharide, disaccharide, trisaccharide, oligosaccharide, polysaccharide, glycoside, glycan, glycosaminoglycan, glycoprotein, glycopeptide, glycolipid, glycolipopeptide, nucleotide, nucleoside, nucleoside phosphate, and nucleic acid.

In some embodiments of the method, the sample is obtained during the production of a recombinant glycoprotein in the pharmaceutical or research industries.

In some embodiments of the method, glycosylation profiles are monitored during bioprocessing.

In some embodiments, The method of any one of claims 22 to 42, wherein the sample includes at least one chemically or enzymatically synthesized carbohydrate or carbohydrate containing compound.

In some embodiments, a sample is an environmental or biological sample.

In some embodiments, a sample is or is from a microorganism. In some embodiments, the microorganism is a virus, bacterium, yeast, fungus or protozoan.

In some embodiments, the sample is from a plant or an animal. In some embodiments, the animal is a mammal. In some embodiments, the mammal is a human.

The present invention includes software that the converts one or more intensities measured in a method described herein into a percentage of glycan present in the sample.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### BRIEF DESCRIPTION OF THE FIGURES

FIG. 1 shows a schematic representation of the multiplexed interactions between multiple suspension array technology (SAT) reagents and a glycoprotein analyte. Glycan specific lectins are conjugated to red fluorescent multiplex microspheres (beads), and then incubated with a green fluorescently labeled glycoprotein. The amount of glycoprotein bound to each bead is measured using flow cytometry.

FIG. 2 shows how in flow cytometry particles in a sample are hydrodynamically focused and flow in a single file through a detector, as light scatter and fluorescence emission are measured for each particle.

FIG. 3 shows a conceptual representation of real-time monitoring of glycosylation during protein expression.

FIG. 4 shows a representative scatter dot plot of Multiplexed Suspension Glycoprofiling Array beads (left) and GlcNAcβ1-4GlcNAcβ-PAA-fluorescein bound (right). Bead 1—ethanolamine quenched; Bead 2—SNA I; Bead 3—MAL II; Bead 4—GS II; Bead 5-ConA; and Bead 6—ECA.

FIG. 5 shows specific detection of directly-labeled GlcNAcβ1-4GlcNAcβ-PAA-fluorescein by MSA element GSII, which is specific for terminal GlcNAc. Intensities for beads with no reagent were subtracted.

FIG. 6 shows secondary detection of GlcNAcβ1-4GlcNAcβ-PAA-biotin by MSA element GSII, which is specific for terminal GlcNAc. Intensities for beads with no reagent were subtracted.

FIG. 7 shows secondary detection of Neu5Acα2-6[Galβ1-4GlcNAcβ1-3]2β-Sp-Biotin by MSA element SNA I, which is specific for the terminal Neu5Acα2-6Gal sequence. Intensities for unlabeled (blank) beads were subtracted.

FIGS. 8A and 8B show binding of GM1 (GM1-LC-LC-biotin). Intensities for beads with no reagents were subtracted.

FIGS. 9A and 9B show binding of biotinylated fetuin and asialofetuin glycoproteins. FIG. 9A shows binding of fluorescently labeled fetuin and asialofetuin glycoproteins, average of three experiments. FIG. 9B shown the difference in binding between fetuin and asialofetuin. Intensities for beads with no reagents were subtracted.

### DETAILED DESCRIPTION OF ILLUSTRATIVE EMBODIMENTS OF THE PRESENT INVENTION

The present invention is includes compositions and methods directed to the multiplexed analysis of carbohydrates and carbohydrate containing compounds. As used herein, the phrase “multiplex,” or grammatical equivalents, refers to the simultaneous detection of multiple analytes in a single assay. Multiplexed analysis is the ability to perform multiple discrete assays in a single tube with the same sample at the same time. In some embodiments of the multiplexed assays described herein, two, three, four, five, six, seven, eight, nine, ten, eleven, twelve, thirteen, fourteen, fifteen, sixteen, seventeen, eighteen, nineteen, twenty, or more analytes may be measured. In some embodiments, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty analytes may be measured. In some embodiments, more that two, more than three, more than four, more than five, more that six, more than seven, more than eight, more than nine, more than ten, more than eleven, more than twelve, more than thirteen, more than fourteen, more than fifteen, more than sixteen, more than seventeen, more than eighteen, more than nineteen, or more than twenty analytes may be measured. In some embodiments, about ten, about twenty, about thirty, about forty, about fifty, about sixty, about seventy, about eighty, about ninety, about one hundred, or more analytes may be measured. In some embodiments, at least about ten, at least about twenty, at least about thirty, at least about forty, at least about fifty, at least about sixty, at least about seventy, at least about eighty, at least about ninety, or at least about one hundred analytes may be measured. In some embodiments, more than about ten, more than about twenty, more than about thirty, more than about forty, more than about fifty, more than about sixty, more than about seventy, more than about eighty, more than about ninety, or more than about one hundred analytes may be measured. In some embodiments, hundreds, or thousands of analytes may be measured.

Unlike protein sequences, which are encoded by the organism's genetic material, the subsequent attachment of complex carbohydrates (glycans) in eukaryotes is controlled by enzymes that either trim or extend the glycan core. A single protein frequently exhibits multiple versions of the glycan, depending on the age or location of the protein. Variations in the glycosylation pattern (glycoprofile) can also result from a range of diseases that introduce mutations into gene sequences, or that alter regulatory control pathways. Aberrant protein glycosylation is therefore a hallmark of several disease states, including diabetes (Coppo and Amore, 2004, Kidney International; 65(5):1544-1547), IgA nephropathy (Amore and Coppo, 2000, Nephron; 86(3):255-259), and various cancers (Krengel et al., 2004, J Biol Chem; 279(7):5597-5603). Because of their exposure on cell surfaces, the glycan chains frequently also serve as receptors for viral and bacterial pathogens (Lim et al., 2008 J. Proteome Res. 7(3):1251-63). The ability to characterize glycoprofiles is therefore relevant to disease marker discovery, the development of therapeutics, the study of infectious diseases, and glycobiology research in general.

The compositions and methods described herein utilize suspension array technology (SAT). With suspension array technology, an assay is carried out with the array elements suspended in a liquid or gel phase. The multiplex suspension assays described herein utilize an array of different carbohydrate binding molecules, each carbohydrate binding molecules with a known carbohydrate binding specificity, to obtain a glycoprofile of the carbohydrate structure(s) in a sample. As used herein, the term “carbohydrate,” also referred to herein as “glycan,” is meant to refer to an organic compound of a general formula Cₘ(H₂O)ₙ. Such multiplexed suspension arrays (MSA) will provide for the rapid, robust, and cost-effective characterization of glycosylation patterns. Such multiplexed suspension arrays for the characterization of glycosylation patterns are also referred to herein as “Glycoprofiling Multiplexed Suspension Arrays,” “glycoprofiling multiplexed suspension arrays,” “glycoprofiling multiplexed suspension arrays (MSA),” “glycoprofiling MSA,” “multiplexed suspension arrays glycoprofiling,” “multiplexed suspension arrays (MSA) glycoprofiling,” “MSA Glycoprofiling,” or “GlycoProf MSA™.”

Each carbohydrate binding molecule of a given specificity is linked to the external surface of a population of individually addressable particles. Preferably individually addressable microspheres such as beads or nanoparticles are employed. Normally the surface of each bead is functionalized with a single type of carbohydrate binding molecule, although in some embodiments a bead can be functionalized with two or more types of carbohydrate binding molecules. The array elements in suspension array technology are modular and suspended in a liquid or gel; typically the array elements take the form of individual particles.

By judicious choice of carbohydrate-specific reagents, the glycoprofiling multiplexed suspension arrays described herein provide a simple but robust technology, able to resolve such differences as the termination state of glycan sequences. FIG. 1 shows a schematic representation of the multiplexed interactions between multiple suspension array technology (SAT) reagents and a glycoprotein analyte. In the embodiment shown in FIG. 1, glycan specific lectins are conjugated to red fluorescent multiplex microspheres (beads), and then incubated with a green fluorescently labeled glycoprotein.

The MSA glycoprofiling approach described herein combines suspension array technologies (SAT) with established high-throughput detection. Any of a variety of detection methods and addressable particles may be used, such as, for example, any of those reviewed in more detail in Braekmans et al., 2002, Drug Discovery; 1:447-456; Wilson et al., 2006, Agnew Chen Int Ed; 45:6104-6117; and Birtwell and Morgan, 2009, Integr Biol; 1:345-362 (which are herein incorporated by reference in their entireties).

In some embodiments, binding detection in SAT methods employs target-specific receptors that are conjugated to the surface of microspheres (beads) with distinct optical properties, such as light scatter based, for example, on bead size or granularity, and/or fluorescence from an internal agent. A fluorescent agent includes, for example, a fluorescent dye, quantum dots, and surface-enhanced raman scattering (SERS).

Any of a variety of protein-attachment chemistries may be used for attachment to an addressable particle, ranging from, for example, physical adsorption or covalent coupling, to specific noncovalent attachment using affinity tags (poly-his, biotin, glutathione-S-transferase, etc.).

The binding of a carbohydrate, carbohydrate containing compound, or glycoprotein bound to each bead maybe determined with the use of a secondary binding agent or an affinity partner with a binding specificity for the analyte, carbohydrate, carbohydrate containing compound, or glycopeptide being assayed. Such a secondary binding agent or affinity partner may be detectably labeled, for example a labeled antibody. Such an antibody may be labeled with, for example, a fluorophore, biotin, or an enzyme. A biotin-streptavidin based detection scheme may be used. Fluorophores include, for example, fluorescent dyes such as phycoerythrin (PE), one of the many ALEZA FLUORs, and reactive water soluble fluorescent dyes of the cyanine dye family, such as Cy2, Cy3, or Cy5. See, for example, “Antibody labeling from A to Z,” Invitrogen 2008 (available on the world wide web at invitrogen.com/etc/medialib/en/filelibrary/cell_tissue analysis/pdfs.Par.60486.File.dat/B-075469-Zenon%20Brochure-flr.pdf). Alternatively, the carbohydrate or glycopeptide being assayed may be directly labelled with such a detectable label.

While a variety of detection methods may be employed, including, but not limited to flow cytometry, image based systems, and microscope based systems. In some embodiments, an image based system may be used. Examples include, but are not limited to, Luminex's MAGPIX (see luminexcorp.com/Products/Instruments/index.htm), Amnis's ImageStream (see amnis.com/documents/brochures/ImageStreamx_brochure.pdf) and spectral flow cytometer (see onlinelibrary.wiley.com/doi/10.1002/cyto.a.20706/full), and Nexcelom Biosciences' Cellometer.

In some embodiments, flow cytometry is a preferred detection method. Flow cytometry is a powerful platform for high-throughput and quantitative functional analysis of cells, and of purified proteins and other biomolecules using microspheres. Flow cytometry rapidly measures the fluorescence and other optical properties of individual particles. The basic principles of flow cytometry, as well as the numerous variations, have been well described (Shapiro H M. Practical Flow Cytometry. 4th. New York: Wiley-Liss; 2004). See also, Nolan and Sklar, 1998, Nat Biotechnol; 16: 633-638; Nolan et al., 2006, Curr Protoc Cytom; Chapter 13:Unit13.8; Yang and Nolan, 2007, Cytometry A; 71(8):625-31; and Nolan and Yang, 2007, Brief Funct Genomic Proteomic; 6(2):81-90.

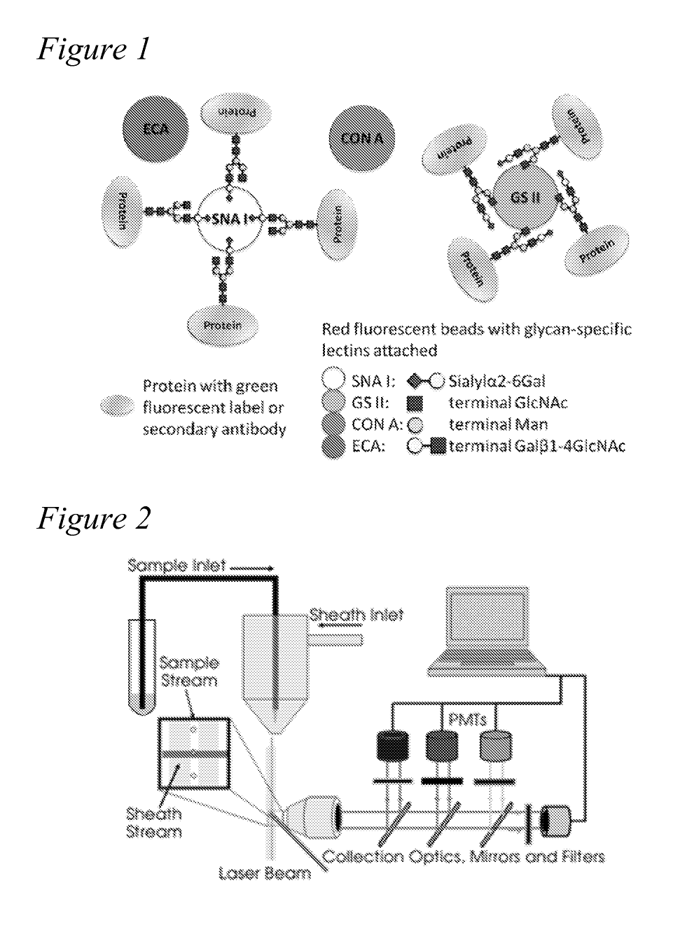

In a typical flow cytometer (FIG. 2), sample is carried in a sheath stream through a laser beam where fluorescent dyes are excited. The emitted fluorescence is collected, spectrally filtered and detected using photomultiplier tubes. Flow cytometry provides for high speed single particle analysis and selection. Samples are hydrodynamically focused to a very thin sample stream, typically on the order of 10 μm in diameter. This focused sample stream is passed through a focused laser beam on the order of 10 μm in height. The intersection of the sample stream and laser beam (FIG. 2, inset), often called the probe volume, has dimensions of ˜10 μm³, or about 1 pl. Under these conditions, in a typical mammalian cell (diameter˜10 μm) suspension, cells will be lined up single file and will pass one at a time through the probe volume, where fluorescence and light scatter signals are collected. Typical transit times through the probe volume are 10 μs or less for many commercial flow cytometers, enabling sample analysis rates of thousands of cells or beads per second. High speed cell sorters are capable of analysing tens of thousands of cells or beads per second (Ibrahim and van den Engh, 2003, Curr Opin Biotechnol; 14:5-12), and sorting selected sub-sets of cells or beads into tubes or microwell plates. Because the measurement probe volume is small, background signal, which often limits sensitivity, is low, making flow cytometry an especially sensitive fluorescence detection platform. While custom instruments have reported single molecule sensitivity (Keller et al., 2002, Anal Chem; 74:316A-324A; and Habbersett and Jett, 2004, Cytometry A; 60:125-34.3), most commercial cytometers have detection limits of a few hundred molecules of a small organic fluorophore such as fluorescein. Intensity standards and calibration protocols have been developed that allow fluorescence measurements to be expressed in absolute units of molecules per cell (Habbersett and Jett, 2004, Cytometry A; 60:125-34; Schwartz et al., 1996, Cytometry; 26:22-31; Schwartz et al., 1998, Cytometry; 33:106-14; Schwartz et al., 2004, Cytometry B Clin Cytom; 57:1-6; Wood and Hoffman, 1998, Cytometry; 33:256-9). These approaches consider instrument response, the properties of reagents used (the fluorophore to protein ratio of an antibody, for example), and spectral matching between calibrators and unknowns. Such absolute quantification facilitates assay development and mechanistic studies, and is critical for certain clinical applications.

Flow cytometry can make high speed, quantitative optical measurements of multiple fluorophores simultaneously. The simplest bench top instruments typically measure three or four colors of fluorescence excited by a single laser. Additional lasers and detectors enable the detection of additional fluorophores, and the past decade has seen a steady increase in the number of parameters measured (De Rosa et al., 2001, Nat Med; 7:245-8; Roederer et al., 1997, Cytometry; 29:328-39), such that three laser eight color experiments are not uncommon, and 19 parameter (fluorescence plus light scatter) measurements have been reported (Perfetto et al., 2004, Nat Rev Immunol; 4:648-55). The high information content provided by multiparameter measurements not only allows for more efficient analysis of samples, it is required to identify key sub-populations present in a complex mixture of cells. Because the probe volume in the flow cytometry measurement is small, signal from free fluorophore is often negligible, allowing samples to be measured without a wash step. In addition, homogeneous assays enable continuous kinetic resolution, allowing flow cytometry to be exploited for real-time mechanistic studies of biochemical processes. Such wash-less assays enable streamlined sample processing and are especially amenable to automated analysis.

Cytometric measurements (fluorescence channel) may be calibrated in terms of mean equivalent soluble fluorescein molecules (MESF) using calibrated FITC-labeled microspheres. Standard curves may be generated. Commercial software is available to for assist with data analysis. The prototypical multiplexed bead-based analysis is the antibody sandwich assay. Essentially, an ELISA performed on a microparticle instead of a microwell bottom, an immobilized antibody captures an analyte from a complex sample, and a labeled reporter antibody completes the sandwich allowing the analyte to be quantified via the fluorescence intensity of the microsphere. The principles and considerations for developing such multiplexed assays have been described in detail (Camilla et al., 2001, Clin Diagn Lab Immunol; 8:776-84; Carson and Vignali, 1999, J Immunol Methods; 227:41-52; Kellar et al., 2001, Cytometry; 45:27-36). In general, the bead-based assays offer sensitivity comparable to the standard colorimetric ELISA, with the advantages of smaller sample size, fewer processing steps, which combined with the efficiency of multiplexing constitute an extremely powerful approach to the detection of soluble proteins.

In terms of convenience, and cost, it is important to note that the most basic benchtop flow cytometers, with one or two lasers and four or five detectors, are capable of making sensitive (a few hundred to a few thousand molecules) and quantitative measurements of multiple different fluorescent probes simultaneously on individual particles. In the presently available systems, a 100-plex SAT assay can be performed approximately every 30 seconds. Current flat array technologies employ 11-45 target-specific receptors, which is within the current dynamic range of flow cytometry based SAT. Running continuously, such systems could process 288,000 assays per day, allowing the MSA glycoprofiling approach described herein to be used for real-time process monitoring, as well as for the analysis of large numbers of samples, as for example in a regulatory laboratory.

The MSA glycoprofiling approach described herein may make use of individually addressable particles. Such individually addressable particles include, for example, microspheres and nanoparticles. In preferred embodiments, individually addressable particles are optically encoded microspheres; microspheres with distinct optical properties, such as light scatter or fluorescence from an internal dye. Based on a dye color coded scheme, 100 or more distinct sets of optically encoded microspheres, also referred to as color coded beads, can be produced. Because of the dye ratio incorporated each bead, each unique bead population can be analyzed separately when lasers are used to excite the internal dyes that identify each microsphere particle. Each bead set will have a separate capture reagent, such as a separate carbohydrate binding molecule, attached to the surface, allowing for the capture and detection of specific analytes from a sample. Encoded microspheres and flow cytometry have been employed for a wide range of multiplexed molecular analysis, and detailed protocols for many of these have been developed. See, for example, Fulton et al., 1997, Clin Chem; 43:1749-56; Kettman et al, 1998, Cytometry; 33:234-43; and Oliver et al., 1998, Clin Chem; 44(9):2057-60. Encoded microspheres are commercially available from a number of sources, including, for example, Spherotech (Lake Forest, Ill.).

Each derivatized batch of microspheres (array element) may be prepared in bulk, and by virtue of the solution phase chemistry employed for conjugation, the receptors are dispersed evenly over the surface of the sphere. Because the target-receptors are conjugated to beads, the elements of the array may be combined and altered at will. Arrays with particular reagents may be created that target the interests of a particular research community, a particular pharmaceutical company, or a Federal regulatory body. In addition, SAT analyses may be performed on any flow cytometer (FIG. 2), without the need to dedicate it to SAT use. The use of flow cytometry has some very significant advantages in terms of statistical precision and reproducibility over flat array technologies.

With the MSA glycoprofiling approach described herein, each bead set will have a separate capture reagent, such as a separate carbohydrate binding molecule, attached to the surface, allowing for the capture and detection of specific analytes from a sample. Carbohydrate binding molecules include, but are not limited to, lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains (such as cholera toxin B, other toxins, and hemagglutinin), aptamers including protein, RNA or other small molecule aptamers, and any other molecule that naturally binds or is engineered to bind a carbohydrate.

Lectins are widely used carbohydrate-binding molecules for glycoprofiling. Any of a variety of lectins (sugar-binding proteins), including, but not limited to, any of those described herein, may serve as a carbohydrate binding molecule. Lists of representative carbohydrate binding lectins are also included in the examples provided herewith. Lectins are not, however, ideal reagents. They are not generally high affinity, and some lectins display relatively broad specificity, or context dependency. As an illustration, the lectin MAL II which is known to prefer Sialylα2-3Gal linkages, displays strong context dependence; an examination of the CFG binding data indicates that MAL II will bind to the linear sequence Sialylα2-3Galβ1-4GlcNAcβ1-3Galβ1-4GlcNAcβ1-3Galβ1-4GlcNAcβ, but will not recognize the related branched sequence Sialylα2-3(Galb1-3GalNAcb1-4)Galb1-4Glcb. In direct contrast, the carbohydrate binding B domain from cholera toxin (CTB) binds the branched sequence, but not the linear. The CFG glycan array data provides an unrivalled source of experimental specificities from which to select reagents with well-defined specificities. Thus, to enhance the robustness of a glycoprofiling MSA methods, redundant MSA reagents may be employed, such as the lectins PSL and SNA I, both of which bind to Sialylα2-6Gal linkages.

In addition to lectins, other well-characterized carbohydrate-detection reagents, such as pathogen adhesion domains and antibodies, may serve as carbohydrate binding molecules. A carbohydrate binding molecule may be an antibody with a binding specificity for a carbohydrate determinant. Such antibodies, include, but are not limited to, any of those described herein. Lists of representative carbohydrate binding antibodies and lectins are also included in the examples provided herewith. Anti-carbohydrate antibodies provide an alternative to lectins, but they are also known to display cross-reactivities with dissimilar glycans. For these reasons, reagents with redundant binding properties will be employed for a robust glycoprofiling technology.

One or more of the antibodies or lectins employed as carbohydrate-specific receptors for glycoprofiling with microarrays may be used in the multiplex suspension array glycoprofiling approach of the present invention. See, for example, Chandrasekaran et al., 2002, Glycobiology; 12(3):153-162; Davidson et al., 2000, Hum Pathol; 31:1081-1087; and Prien et al., 2008, Glycobiology; 18(5):353-366.

Carbohydrate binding molecules used in the MSA glycoprofiling approach of the present invention include carbohydrate processing enzymes that have been inactivated but still bind to carbohydrate(s) with high specificity. Such molecules, also referred to herein as a “LECTENZ” molecule, a “Lectenz®” molecule, or a “lectenz,” include a catalytically inactive mutant of a carbohydrate-processing enzyme that has substantially the same specificity for a given glycan as the wild-type enzyme, and an increased affinity towards the glycan as compared to the WT enzyme. As used herein, the term “substantially the same” is meant to describe a specificity of the glycosidase mutant that is at least 60% of the wild-type enzyme. In some embodiments, the specificity of the mutant is at least 70% of the WT enzyme. In at least one embodiment, the mutated glycosidase is at least 85% as specific to its substrate as the wild-type enzyme to the same substrate. In other embodiments, the mutated glycosidase is at least 95% as specific to its substrate as the wild-type enzyme to the same substrate. LECTENZ molecules are based on the directed affinity evolution of inactivated carbohydrate-processing enzymes. As these reagents are derived from enzymes with very-high carbohydrate specificity, they do not suffer from the cross-reactivities frequently exhibited by both lectins and antibodies.

LECTENZ molecules are not limited to any specific carbohydrate processing enzyme. Rather, broadly applicable to any glycosidase or glycosyltrasferase enzyme, protein, or polypeptide capable of specifically recognizing a carbohydrate. Examples of glycosidases suitable for the present inventions include, but are not limited to, lactase, amylase, chitinase, sucrase, maltase, neuraminidase, invertase, hyaluronidase, and lysozyme. Glycosidases of the present invention can be inverting or retaining glycosidases. In one embodiment, a LECTENZ is prepared from PNGase F, isolated from Flavobacterium meningosepticum. In another embodiment, the lectenz is prepared from recombinant B-O-GlcNAcase, with the WT sequence as determined for β-O-GlcNAcase isolated from Bacteroides thetaiotaomicron. In yet another embodiment, neuraminidase from Clostridium perfringens is used to prepare a LECTENZ. In addition to glycosidases, carbohydrate-processing enzymes suitable for use in the present invention include glycosyltransfeases and polysacharide lyases. Other carbohydrate-processing enzymes include carbohydrate esterases, sulfatases, sulfotransferases, or any other enzyme that acts on a carbohydrate substrate. Catalytically inactive carbohydrate-processing enzymes of the present invention can be prepared from carbohydrate-processing enzymes isolated from prokaryotic or eukaryotic organisms, as well as others.

In certain embodiments, the carbohydrate-processing enzyme is a glycosidase enzyme. In other embodiments, the carbohydrate-processing enzyme is a glycosyltransferase enzyme. In other embodiments, the carbohydrate-processing enzyme is a polysaccharide lyase enzyme. In other embodiments, the carbohydrate-processing enzyme is a sulfatase enzyme. In other embodiments, the carbohydrate-processing enzyme is a sulfotransferase enzyme. In other embodiments, the carbohydrate-processing enzyme is a ligase enzyme. In further embodiments, the carbohydrate-processing enzyme is an amidase enzyme. In yet further embodiments, the carbohydrate-processing enzyme is an epimerase enzyme.

Representative carbohydrate-processing enzymes that can be used to form LECTENZ molecules suitable for use in the multiplexed assay of the invention include, without limitation, glycosidase enzymes, glycosyltransferase enzymes, polysaccharide lyase enzymes, sulfatase enzymes, sulfotransferase enzymes, ligase enzymes, amidase enzymes, and epimerase enzymes. Examples of LECTENZ molecules that make useful array elements include LECTENZ molecules derived from PNGase F (an amidase) and LECTENZ molecules derived from O-GlcNAcase.

See WO2010/068817 (“Glycan-Specific Analytical Tools,” published Jun. 17, 2010), which is incorporated by reference herein in its entirety, for a more complete description of LECTENZ molecules.

A multiplexed suspension array according to the invention can be formed exclusively from lectins, antibodies or LECTENZ molecules; however it is expected that multiplexed arrays that incorporate multiple types of carbohydrate binding antibodies, such as both lectins and LECTENZ molecules, or both antibodies and lectins, or both antibodies and LECTENZ molecules, or all three types of carbohydrate binding molecules, with or without any other typed of carbohydrate binding molecules, will provide a more useful platform for glycoprofiling, as it will help to increase the certainty of identification of a particular glycan if one or more of the carbohydrate binding molecules that bind that glycan exhibit cross-reactivity with other glycans.

The MSA glycoprofiling approach of the present invention provides many advances and advantages over currently used technologies, including, MS, microplate assays, and solid phase microarrays. Some advantages include, but are not limited:

Storage and Handling is improved. Array elements have a long shelf life (>6 months at 4° C.), because the array elements are stored in buffer until use.

The addition on new elements is simplified. A suspension of microspheres typically contains tens of millions of particles per milliliter that, when coupled with the appropriate receptor can be used to prepare thousands of microsphere arrays. To reconfigure an array with new array elements, a new conjugation is performed on a particular microsphere subset and a new mixture of microspheres is prepared.

Array density is greatly increases. While the current generation of suspension arrays contain between a dozen and a hundred discrete array elements, optical encoding approaches make very high-density arrays possible.

Statistical reproducibility is improved. A few microliters of microspheres typically contain tens of thousands of array elements. Thus each element in the array is represented by several hundred individual microspheres, thus the flow-cytometric measurement represents a replicate analysis of each array element.

Throughput is increased. Using flow cytometry as a measurement platform, particle analysis rates can be as high as 10,000 s-1, making highly multiplexed analysis extremely rapid.

Ligand binding kinetics and thermodynamics are improved. The process is an equilibrium process, therefore making it possible to determine KA values. Liquid reaction kinetics gives faster, more reproducible results than with solid, planar arrays.

The approach is driven by increasing demand for analytical methods to measure large numbers of biomolecules quantitatively and sensitively in small volumes of sample.

Reduced cost and labor is obtained by multiplexing.

There is a shortened time to results by favorable reaction kinetics of liquid bead array approach, with smaller sample requirements.

A further advantage of the suspension array technology used with the present invention, both in terms of throughput and accuracy, is that, whereas procedures using flat microarrays often require extensive washing to reduce high background signals, the ability of flow cytometry to resolve free and bound probes enables assays to be performed with minimal or no wash steps, streamlining sample processing. In the particular case of glycoprofiling, it is notable that, lectins generally have low affinity for their carbohydrate ligands and the interactions may not be able to survive the extensive washing steps (Horimoto and Kawaoka, 2005, Nat Rev Microbiol; 3(8):591-600).

The ability to perform multiplexed analyses of suspension arrays, in small sample volumes, for many target glycoprotein samples, makes the MSA glycoprofiling approach described herein a powerful alternative to less flexible flat surface arrays. By combining this technology with common and established cytometry instrumentation, there is a potential to make an almost immediate impact on the manner in which glycosylation analyses are performed. This approach should open the field of glycoprofiling up to laboratories that would otherwise find such analyses daunting, and should provide a tool to meet the unmet needs for real-time process control in the production of therapeutic glycoproteins.

Advantageously, the multiplexed suspension assay can include particles (array elements) with overlapping or redundant specificities, which can increase the level of confidence in the data obtained when analyzing or characterizing a carbohydrate containing sample.

It should be understood that the particular array elements used in the multiplexed suspension array technology are selected based upon the research or clinical interest of the user; indeed, the ability to formulate, in a modular fashion, a customized set of array elements is what imparts the unique flexibility to this technique. It is not possible to set forth herein every possible combination of array elements that might be of interest to a user nor should it be necessary, as one of skill in the art can readily imagine a vast number of permutations and can create a custom array of any number of array elements by functionalizing the desired number of beads with the desired number and type of carbohydrate binding molecules.

The present invention includes compositions and methods including any combination or subcombination of specific carbohydrate binding molecules described herein; for example, any two, any three, any four, any five, any six, any seven, any eight, any nine, any ten, any eleven, any twelve, any thirteen any fourteen, any fifteen, any sixteen, any seventeen, any eighteen, any nineteen, any twenty, or more of the a specific carbohydrate binding molecule described herein.

In some embodiments, the binding of the carbohydrates or carbohydrate containing compounds to the functionalized particles is conveniently detected or monitored using fluorescence-based techniques such as flow cytometry; however, other detection techniques are envisioned which may encompass both batch and flow process, and are selected based on the type of labeling agent used for the microspheres and/or the carbohydrate or carbohydrate containing compound (fluorescent, phosphorescent, magnetic, electromagnetic, radioactive, enzymatic, and the like). For example, any of the various detection methods and addressable particles reviewed in more detail in Braekmans et al., 2002, Drug Discovery; 1:447-456; Wilson et al., 2006, Agnew Chen Int Ed; 45:6104-6117; and Birtwell and Morgan, 2009, Integr Biol; 1:345-362 (which are herein incorporated by reference in their entireties) may be used.

Carbohydrates and carbohydrate containing compounds that can be detected using the multiplexed suspension assay of the invention include but are not limited to disaccharides, trisaccharides, oligosaccharides, polysaccharides, glycosides, glycans, glycosaminoglycans, glycoproteins, glycopeptides, glycolipids, glycolipopeptides, nucleotides, nucleosides and nucleic acids. A carbohydrate can include a monosaccharide, a disaccharide or a trisaccharide; it can include an oligosaccharide or a polysaccharide. An oligosaccharide is an oligomeric saccharide that contains two or more saccharides and is characterized by a well-defined structure. A well-defined structure is characterized by the particular identity, order, linkage positions (including branch points), and linkage stereochemistry (α,β) of the monomers, and as a result has a defined molecular weight and composition. An oligosaccharide typically contains about 2 to about 20 or more saccharide monomers. A polysaccharide, on the other hand, is a polymeric saccharide that does not have a well defined structure; the identity, order, linkage positions (including brand points) and/or linkage stereochemistry can vary from molecule to molecule. Polysaccharides typically contain a larger number of monomeric components than oligosaccharides and thus have higher molecular weights. The term “glycan” as used herein is inclusive of both oligosaccharides and polysaccharides, and includes both branched and unbranched polymers. When a carbohydrate contains three or more saccharide monomers, the carbohydrate can be a linear chain or it can be a branched chain.

Larger carbohydrate containing structures can also be detected using the multiplexed suspension assay of the invention. Examples of larger detectable structures include cell membrane components and cell wall components, components of an extracellular matrix, virions, virus particles, and partial or whole virus or partial or whole cells, including bacteria, yeast, protozoans and fungi.

Applications (and associated markets) of the glycoprofiling platform described herein include the characterization of isolated glycoproteins and the monitoring of glycosylation during glycoprotein expression.

Research groups and regulatory agencies need to characterize the glycoprofiles of specific, purified glycoproteins. The glycoprofiling platform described herein addresses this by providing insight into the relative levels of the terminal glycan components that define unique sequences associated with glycosylation. In addition, by careful choice of the carbohydrate-receptor proteins in the array, the linkages and configurations between the monosaccharides that comprise the glycans can be determined. This information will enable a researcher to elect whether or not to pursue more detailed analysis by MS. Moreover, when the carbohydrate-receptor protein is a reagent, such as a diagnostic antibody, the glycoprofiling platform described herein will be extremely useful in the screening of samples for the discovery of glycoproteins that carry disease marker glycans. The role of glycans in biological development and disease makes them obvious targets for detection, diagnostic, and therapeutic applications. A lack of sufficient glycan-specific analytical tools is responsible in part for the delay in fully exploiting aberrant glycosylation in the diagnosis and treatment of disease. There is an urgent need for biosensors with defined carbohydrate specificity that can be used to interrogate biological samples in the search for abnormal glycosylation. In a 2007 White Paper Report from Focus Groups at the NIH Workshop on Frontiers in Glycomics and Glycobiology, it was concluded that: “The analytical technology available for the specific analysis of glycoconjugates is lagging behind that of the technologies available to the scientific community for the study of genomics and proteomics and their function in disease and assigns the highest priority to the support of the development of glycan-specific analytical tools.”

From the perspective of a regulatory agency, or biopharmaceutical company, the glycoprofiling platform described herein provides a method for fingerprinting the glycosylation state, which would serve a key role in identifying batch variations in therapeutic glycoproteins. Such variations routinely occur, for example when a new cell-type is employed for expression, and may even arise from minor differences in growth medium.

Another major application for rapid glycoprofiling technologies is real-time monitoring of the glycosylation state of a protein during glycoprotein production. This need is unmet by existing technologies. An essential regulatory requirement in the commercial production of glycoproteins is maintaining uniform glycosylation profiles. Given that industrial fermentation scales may be up to 20,000 L per batch, post-production sample failure is an enormously costly event. The alternative industrial production mode, continuous flow, would equally benefit from real time glycoprofiling capability, particularly in that if variations in the glycoprofile were detected, the production stream could be diverted without contaminating the entire batch. Currently, it takes several weeks (months on occasion) to obtain protein quality data. As a result, it is difficult to efficiently incorporate these findings in routine process development

The multiplexed suspension assay described herein is especially useful in methods of glycoprofiling, including real-time analysis during synthesis of carbohydrate containing molecules, as described in more detail below. The multiplexed suspension assay described herein can provide complementary data to that from mass spectrometry (MS)-based methods. While not supplanting more precise techniques for final quality control, multiplexed suspension assay described provides a convenient method for monitoring glycosylation. Notably, the most sensitive methods, such MS are unable to directly determine the linkage type (1-2, 1-3, 1-4, etc.) or the anomeric configuration (α- or β-) between the monosaccharides in a glycan. Consequently, the glycoprofiles determined from MS methods always infer the glycan structure based on expected linkages and configurations. While this is adequate for certain portions of the glycan, which are invariant, it is inadequate for assigning the structures of variable regions. In particular, MS-based techniques cannot determine whether a sialylated glycan (a very common eukaryotic modification) terminates in a Sialylα2-3Gal or Sialylα2-6Gal linkage. Terminal sialylation is critical in determining the bioavailability of therapeutic glycoproteins Huang et al., 2006, Proc Natl Acad Sci USA; 103(1):15-20, can regulate protein function, particularly in the case of therapeutic antibodies (Wang et al., 2008, Proc Natl Acad Sci USA; 105(33):11661-11666; Werz et al., 2007, J Am Chem Soc; 129:2770-2771), can be a key virulence factor in pathogenic bacteria (Hakomori, 1984, Arm Rev Immunol; 2:103-26), and the difference between α2-6 and α2-3 linkages is responsible for defining whether pathogens, such as influenza, are transmissible between humans (α2-6) or not (α2-3).

The multiplexed suspension assay described herein can be used in a regulatory role to monitor batch consistency, as well as provide a routine tool for assessing protein glycosylation in a research environment. Providing the ability to rapidly monitor changes in the glycoprofile during glycoprotein expression would enhance the efficient production of commercial therapeutic glycoproteins.

The multiplexed suspension assays described herein have potential use as a method of detection in many areas, including environmental, fermentation, food and medical areas and could be used for in vivo or in vitro sensing in humans or animals. Environmental samples include, but are not limited to, air, agricultural, water and soil.

Glycans have several distinct properties that make them excellent targets for disease biomarkers. Firstly, the location of the glycans on the cell surface makes them the first point of contact of cellular interactions and thus crucial in the control of normal metabolic processes. Cell surface molecules are also strategically exposed for surveillance by the immune system allowing for the potential of immune recognition of abnormal cells. Secondly, specific glycan structures that are not present, or are in low amounts, in normal states proliferate in disease states. And lastly, changes in glycosylation involve many proteins, including those that are highly abundant. Therefore, a single change in a cell's glycosylation machinery can affect many different glycoconjugates.

In some embodiments, a multiplexed suspension assay as described herein can be used to interrogate biological samples in the search for abnormal glycosylation.

In other embodiments, a multiplexed suspension assay as described herein can be used for the detection of a target carbohydrate-based analyte level in biological fluids. Examples of the target analytes include, but are not limited to, endogenously found molecules, such as N- or O-linked glycans, glycosaminoglycans (including heparin), exogenously consumed species, such as plant polysaccharides, carbohydrate-based drugs, and pathogens, whose surfaces are often coated in complex distinct glycans.

Examples of biological samples include, but are not limited to, any biological fluid, tissue, or organ. Examples of the biological fluids include, but are not limited to blood, urine, serum, lymph, saliva, cerebra-spinal fluid, anal and vaginal secretions, perspiration and semen, of virtually any organism, with mammalian samples being preferred and human samples being particularly preferred.

In some specific embodiments, a multiplexed suspension assay as described herein can be used for diagnosing, and/or treating diseases manifested by abnormal glycosylation. Glycans can regulate different aspects of tumor progression, including proliferation, invasion and metastasis. Changes in glycosylation patterns have been observed in cancers including prostate cancer, colorectal cancer, and breast cancer. Glycoproteins have also provided an ideal source for discovering biomarkers for disease detection. A multiplexed suspension assay as described herein may be useful to identify potential biomarkers in cancer.

In other embodiments, a multiplexed suspension assay as described herein can be used in drug discovery and the evaluation of the biological activity of new glycan-based compounds.

The present invention includes kits including one or more of the compositions described herein, each composition having individually addressable particles; each individually addressable particle having an external surface and having linked to said external surface a separate carbohydrate binding molecule; and each individually addressable particle separately labeled with a detectable label. Each composition may be contained in a separate container or package. A kit may further include one or more secondary binding agents, with a binding specificity for an analyte. A kit may further include one or more reagents for directly labeling the analyte with a detectable label. A kit may further include packaging materials and/or instructions for use. A kit may further include positive and/or negative analyte controls. A kit may be formulated for research, industrial, medical, or veterinary use. A kit may be formulated for flow cytometry analysis. A kit may be formulated for image based analysis. A kit may further include one or more software components to assist in the calculation of relative glycan proportions in a sample.

A software component may assist, for example, in calculations glycan proportions, relative glycan compositions, and/or percentages of a given glycan determinant in a sample. In some embodiments, a software application as described herein is sold separately.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein is applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein.

The present invention and/or one or more portions thereof include circuitry that may include a computer system operable to execute software to provide for the determination of glycan composition. Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and most any device capable of providing suitable computing capabilities may be used according to the present invention. Further, various peripheral devices, such as a computer display, a mouse, a keyboard, memory, a printer, etc., are contemplated to be used in combination with a processing apparatus in the computer system. In view of the above, it will be readily apparent that the functionality as described herein may be implemented in any manner as would be known to one skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Preparation of Beads

Multiplex beads were purchased from Spherotech (Lake Forest, Ill.). Lectins were purchased from Vector Labs and EYLabs and conjugated to the beads using standard coupling chemistry with EDC (1-ethyl-3-[3-dimethylaminopropyl]carbodimide hydrochloride) and Sulfo-NHS (N-hydroxysulfosuccinimide). Glycans will be obtained from commercially available sources. In a typical assay, 200 nM carbohydrate solutions are preincubated with 50 nM SA-Alexa Fluor 488 for 30 minutes in 50 μL total volume. 20,000 of each bead is added and incubated for 30 minutes. The beads are then washed and fluorescence intensity measured by flow cytometry. Binding analyses will be performed as described previously (Nolan et al., 2006, Curr Protoc Cytom; Chapter 13:Unit13.8; Yang and Nolan, 2007, Cytometry A; 71(8):625-31; Nolan and Yang, 2007, Brief Funct Genomic Proteomic; 6(2):81-90).

For standardization of bead preparations the performance of each batch of MSA reagents will be confirmed by using reference glycans, such as those presented in Table 1. The minimum signal/noise (S/N) ratio that permits reliable identification of the binding event will be determined and employed as a lower limit for MSA batch acceptability.

TABLE 1
Array elements and associated reference glycan.

| MSA Reagent | Biotinylated glycans |
|---|---|
| Sambucus nigra lectin I (SNA-I) | Neu5Acα2-6[Galβ1-4GlcNAc |
| | β1-3]₂β- |
| Polyporus squamosus (PSL) | |
| Maackia amurensis lectin II | Neu5Acα2-3[Galβ1-4GlcNAc |
| (MAL II) | β1-3]₂β- |
| Maackia amurensis lectin (MAA) | |
| Griffonia simplicifolia lectin II | GlcNAcβ- |
| (GS II) | |
| Conconavalin A (ConA) | Manα- |
| Erythrina cristagalli lectin (ECA) | [Galβ1-4GlcNAc β1-3]₂β- |
| Cholera toxin B subunit (CTB) | Neu5Acα2-3[Galβ1-3GlcNAcβ1- |
| | 4]Galβ1-4Glcβ- |

To demonstrate the ability of the glycoprofiling MSA approach to quantify glycan binding affinity in terms of equilibrium binding constants, titration of the standard glycans will be performed in the Glycoprofiling Multiplex Suspension Array to generate binding curves from which apparent dissociation constants for the glycans will be determined.

### Example 2

### Glycoprofiling with Multiplexed Suspension Arrays to Distinguish Between Two Glycosylation Sequences

This example assessed the performance of the Glycoprofiling Multiplexed Suspension Array (MSA) with standardized samples of glycans. A multiplexed suspension array (MSA) was prepared by conjugating a subset of five lectins (See Table 2) with known specificities to multiplex microspheres (FIG. 4). Glycans with known structures were obtained from the CFG and assayed for binding to the MSA lectins employing flow cytometry. Unconjugated microspheres or microsphere conjugated to a nonspecific protein may also be used as negative controls.

TABLE 2
Carbohydrate-specific reagents

| Microsphere | MSA Reagent | Specificity |
|---|---|---|
| 1 | Ethanolamine quenched | Negative control |
| 2 | Sambucus nigra lectin I (SNA-I) | Neu5Acα2-6Gal |
| 3 | Maackia amurensis lectin II (MAL II) | Neu5Acα2-3Gal |
| 4 | Griffonia simplicifolia lectin II (GS II) | Terminal GlcNAc |
| 5 | Concanavalin A (ConA) | Terminal Man |
| 6 | Erythrina cristagalli lectin (ECA) | Galβ1-4GlcNAc |

The ability of the MSA glycoprofiling arrays to distinguish between two glycosylation sequences was compared, and, in addition, both direct and secondary detection methods were tested (Table 3).

TABLE 3
Glycans

| Glycan | Glycan Analyte | Spacer | Fluorophore | Role |
|---|---|---|---|---|
| 1 | GlcNAcβ1-4GlcNAcβ-Sp- | Multivalent | Fluorescein | Positive Control |
| | | NHCOCH₂NH | | |
| | | PAA | | |
| 2 | GlcNAcβ1-4GlcNAcβ-Sp-Biotin | Multivalent | Streptavidin-Alexa | Positive Control |
| | | NHCOCH₂NH | Fluor 488 | |
| | | PAA | | |
| 3 | Neu5Acα2-6[Galβ1-4GlcNAcβ1- | Monovalent | Streptavidin-Alexa | Positive Control |
| | 3]2β-Sp-Biotin, also known as 6′S- | Sp-NH-LC-LC | Fluor 488 | |
| | Di-LN | | | |
| 4 | Neu5Acα2-3[Galβ1-3GlcNAcβ1- | Monovalent | Streptavidin-Alexa | Positive Control |
| | 4]Galβ1-4Glcβ-Sp-Biotin, also | Sp-NH-LC-LC | Fluor 488 | |
| | known as GM1 | | | |

Microspheres exist with sufficient fluorescence dynamic range to permit the routine multiplexed analysis of up to approximately 100 unique elements. Illustrated in FIG. 4 is a typical data set from the multiplexed cytometric analysis of the six component MSA Glycoprofiling assay, showing the free and bound bead states.

Direct detection of PAA-conjugates (a model for the analysis of directly-labeled high avidity glycoproteins). GlcNAcβ1-4GlcNAcβ-PAA-fluorescein (Table 3, Glycan 1) is a synthetic polymer, in which the carbohydrate is displayed in a multivalent format that is similar to a high-avidity biological context. The amide groups of the polymer chain were N-substituted with the sugar in a 4:1 ratio, and with fluorescein in a ratio of 100:1. By virtue of it being chemically conjugated to fluorescein, beads that bind to this polymer may be directly detected in the cytometer. Direct labeling could similarly be employed for the analysis of purified glycoprotein samples, but might not be suitable for in-process monitoring, in which the laborious step of isolation and purification should be avoided.

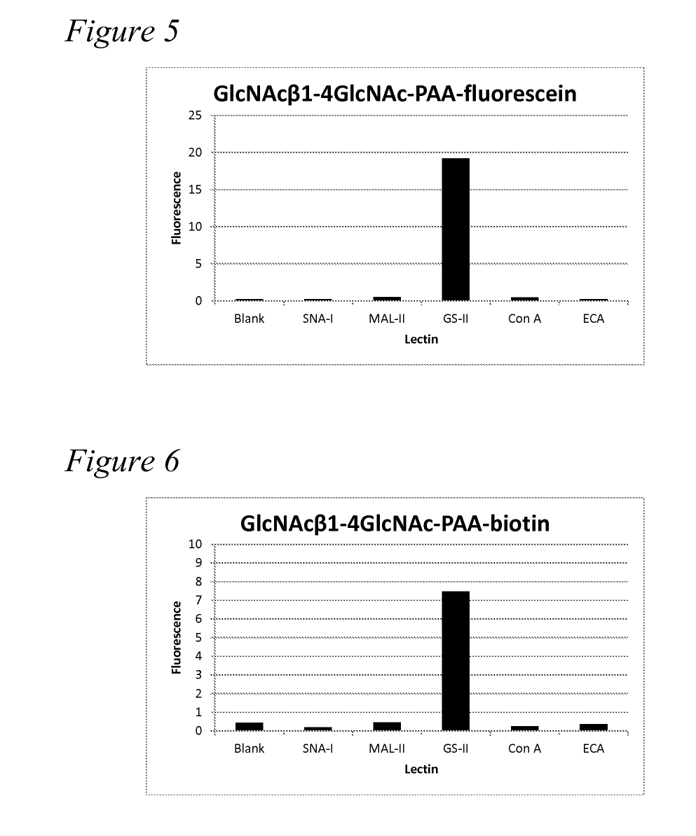

As seen in FIG. 5, the multiplexed analysis gave an excellent signal to noise ratio (S/N>20:1) for all of the detected elements. The GlcNAcβ1-4GlcNAcβ-PAA-conjugate bound to the MSA bead conjugated to GS II, which is a lectin specific for terminal GlcNAc. None of the other MSA elements, including the negative control bound to this glycan. Due to the relatively high concentration of glycans, the PAA conjugates represent a biological context that might be present for example on a mammalian or bacterial cell surface.

Secondary detection of PAA-conjugates (a model for the analysis of unlabeled high avidity glycoproteins). GlcNAcβ1-4GlcNAcβ-PAA-biotin (Table 3, Glycan 2) is also a synthetic polymer. As in the PAA-fluorescein system, the amide groups of the polymer chain were N-substituted with the sugar in a 4:1 ratio, although with biotin in a ratio of 20:1. In contrast to the case of PAA-fluorescein, the biotinylated polymer is used together with a streptavidin Alexa Fluor 488 conjugate for detection. The biotinylated carbohydrate polymer was preincubated with streptavidin-Alexa Fluor 488 in a 4:1 ratio and subjected to analysis (FIG. 6).

A secondary detection step was employed to mimic the application to unlabeled glycoproteins, as in the application of in-process glycoprofile monitoring. In the more general bioprocess case, secondary detection would be performed with an antibody specific for the target glycoprotein. If such an antibody were not be available, direct labeling would be an alternative. However in the commercial production of recombinant glycoproteins, specific antibodies are routinely employed for characterization.

As in the case of direct detection, MSA Glycoprofiling employing secondary detection with labeled-streptavidin correctly identified the glycan as terminating in GlcNAc. It is notable that the signal to noise was again excellent (S/N>10:1). Based on the PAA studies, either direct or secondary detection methods appear to be effective.

Secondary detection of biotinylated glycans (a model for the analysis of unlabeled low abundance glycoproteins). Unlike the PAA-conjugates, most glycoproteins will have lower levels of glycosylation, for example the therapeutic glycoprotein erythropoietin has three N-linked and one O-linked glycosylation positions. Terminal sialylation is critical to the activity and serum half life of therapeutic recombinant glycoproteins, such as human erythropoietin (EPO; the 3D structure of EPO can be found, for example, on the World Wide Web at glycam.org), and so we selected a glycan (Table 3, Glycan 3) that contained a terminal Neu5Acα2-6Gal sequence for analysis.

In addition, in order to assess the performance of the MSA glycoprofiling assay with glycans in a low avidity interaction typical of this type of glycoprotein, the use of the PAA polymer was eliminated. Instead, the monomeric-biotinylated glycan (the SpLCLC spacer is monomeric) was employed. And to mimic the case of bioprocess glycoprofiling, the streptavidin secondary detection system was retained.

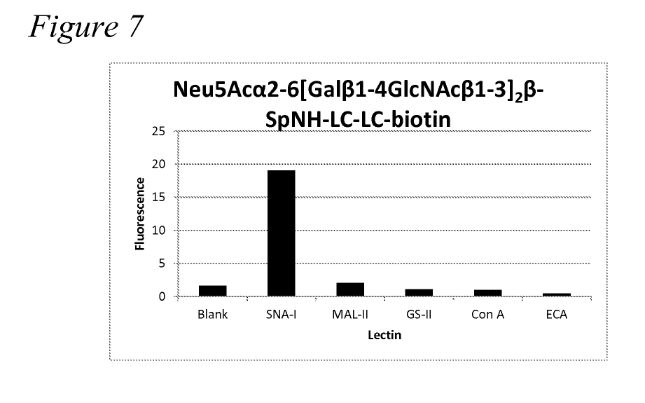

The results for Glycan 3 (FIG. 7) indicate that 6′S-Di-LN bound specifically to MSA bead SNA I, which is specific for Neu5Acα2-6Gal (Table 2). Negligible binding to any of the other MSA elements, including the ethanolamine quenched (blank) control beads, was seen. The signal to noise was again in the range of S/N10:1.

### Example 3

### Multiplexed Suspension Array

### Materials

Activation buffer: 0.1 MES, 0.5 M NaCl, pH.6.0
Coupling buffer: 0.1 M Sodium phosphate, 0.15 M NaCl, pH 7.4
Wash buffer: PBS/0.02% Tween20

### Ice Bucket, Ice

### SPHERO™ Carboxyl Flow Cytometry Multiplex Bead Assay Particles (1×10⁸/ml)

Lectin (1 mg/ml)
EDC (1-ethyl-3-[3-dimethylaminopropyl]carbodimide hydrochloride) (191.7 g/mol)
Sulfo-NHS (N-hydroxysulfosuccinimide) (217.14 g/mol)

### Carbohydrates:

100 μM GM 1-biotin

100 μM 3′S-Di-LN-LC-LC-biotin (2,3)

100 μM 6′S-Di-LN-LC-LC-biotin (2,6)

### Lectin Solutions

SNA-I (2 mg) was resuspended in 2 mL of solution having 0.01 M phosphate, 0.15M NaCl, and 0.05% sodium azide at pH7.4.

GS-II (1 mg) was resuspended in 1 mL of solution having 0.01 M phosphate, 0.15M NaCl, 0.5 mM CaCl₂, and 0.05% sodium azide at pH 7.4.

### Conjugation of Protein to Microsphere

In brief, the bead conjugation was performed using standard EDC/NHS chemistry. Alternatively, the carboxyl groups of proteins can be conjugated to amino microspheres using the same chemistry.

100 μl of microspheres in PBS were placed in a microfuge tube with 325 μL of activation buffer. EDC was dissolved at 100 mg/ml, 522 mM (20 mg in 0.2 mL) in activation buffer. Sulfo-NHS was dissolved at 100 mg/ml, 460 mM (20 mg in 0.2 mL) in activation buffer. 20 μl EDC and 55 μl Sulfo-NHS were then added to each tube. The tubes were incubated for 15 minutes at room temperature.

Following the incubation, the tubes were washed with 1× coupling buffer by spinning at 10000×g for 5 minutes then removing supernatant.

0.1 mL (100 μg) of lectin was added to each tube and 0.4 mL coupling buffer was added to each tube. The tubes were incubated 1 hour at 4° C. with mixing.

The tubes were washed 2× with wash buffer as described above. The remaining pellet was resuspended in 500 μL (2×10⁷/mL) PBS.

### Binding of Carbohydrates to the Protein-Conjugated Microspheres

In brief, biotinylated glycans were incubated with the lectin-conjugated beads, washed, and then detected by a fluorophore labeled streptavidin. Alternatively, the biotinylated glycans can be preincubated with the streptavidin-fluorophore conjugate in a 4:1 ratio.

Standard glycoproteins were biotinylated and measured the same way. Directly labeled glycoproteins or fluorescent antibodies against glycoproteins can also be used.

1 μL of each bead was mixed together. A total bead mixture equivalent to 1 μL of each type of beads were added to 50 μL 50 nM biotinylated carbohydrate. Samples were incubated for 1 hour with occasional vortexing. The supernatant was removed. Beads were washed 1× with 0.5 mL buffer by spinning at 3000 rcf/3 minutes/25° C. and removing supernatant. The beads were resuspended in 50 μL Streptavidin-Phycoerythrin (SA-PE) (1:200 dilution of a 1 mg/1 ml solution) in PBS. In later experiments SA-Alexa Fluor 488 was used. Samples were incubated for 1 hour with occasional vortexing. The supernatant was removed. The beads were washed 1× with 0.5 mL buffer by spinning at 3000 rcf/3 minutes/25° C. and removing supernatant. Beads were resuspended in 200 μL PBS buffer immediately before flow cytometry.

### Results

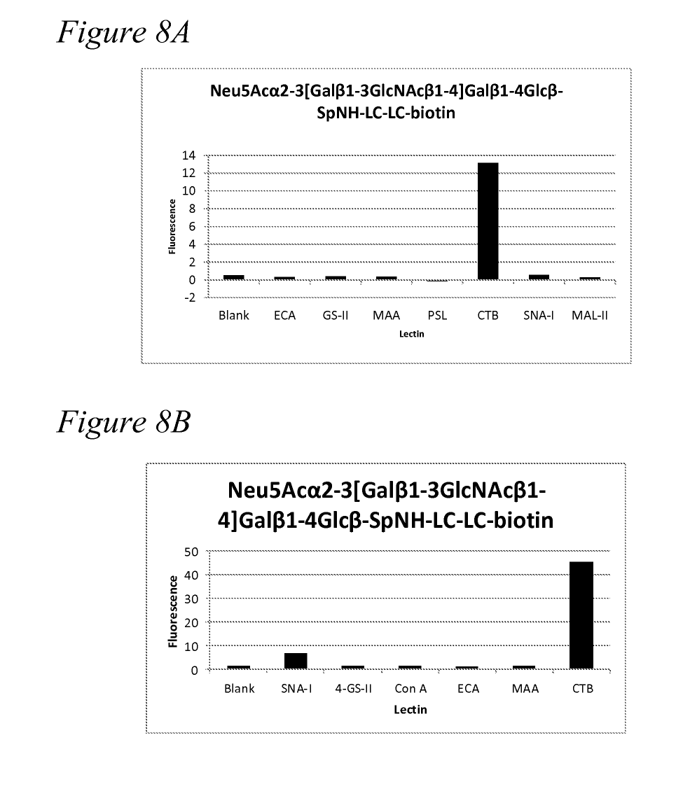

Specificity of the reagents in a multiplexed glycoprofiling suspension array is indicated by the data in FIGS. 8A and 8B, which shows that a glycan containing Neu5Acα2-3[Galβ1-3GlcNAcβ1-4]Galβ1-4Glcβ-(GM1) bound only to the protein cholera toxin B subunit (CTB). CTB is known to be specific for glycans containing GM1. None of the other proteins included in this initial array bound to the GM1 glycan.

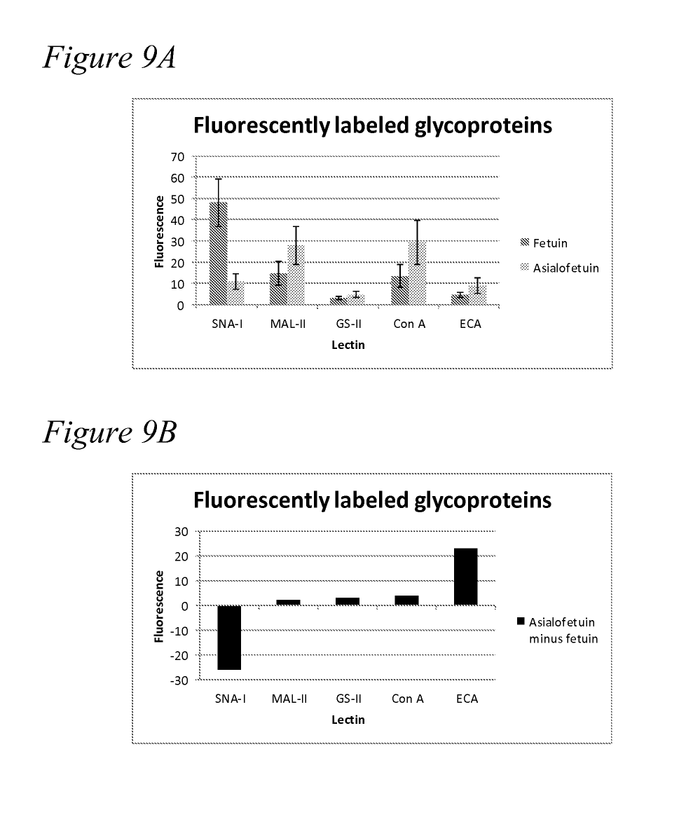

The ability of the reagents to detect glycosylation in glycoproteins (fetuin and asialofetuin) is demonstrated in FIGS. 9A and 9B, which demonstrates that treatment of the glycoprotein (fetuin) with neuraminidase (also known as sialidase) results in the formation of asialofetuin. Treatment with sialidase decreases the amount of sialic acid (also known as Neu5Ac) present in the glycoprotein, revealing terminal galactose. The loss of terminal sialic acid upon treatment of fetuin with sialidase is indicated by the decrease in the binding signal from the protein SNA I, which is specific for glycans containing terminal 2,6 linked sialic acid. The resulting exposure of terminal galactose is indicated by an increase in the binding signal from the protein ECA, which is specific for terminal galactose.

### Example 4

### Glycoprofiling

Glycoprofiling of Isolated Glycoproteins. MESF (Molecules of Equivalent Soluble Fluorochrome) microspheres quantitate the level of glycosylation in microspheres. Commercially available microspheres (for example, purchased from Bangs Labs; Fishers, Ind.) may be used. The MESF value of a bead equals the fluorescence intensity of a given number of pure fluorochrome molecules in solution. For example, an Alexa Fluor 488 microsphere with an MESF value of 10,000 has the same fluorescence intensity as a solution containing 10,000 Alexa Fluor 488 molecules. An MESF kit contains a set of microspheres with discrete levels of fluorochrome. By plotting each population's fluorescence intensity versus the MESF, a standard curve is generated. Such a relationship enables the linearity of the instrument to be confirmed, and the MESF value of the MSA bead can be extrapolated based on this standard curve. Using the MESF value of the MSA bead and the degree of labeling of the glycoprotein, the absolute number of glycoprotein molecules bound to each MSA bead can be determined.

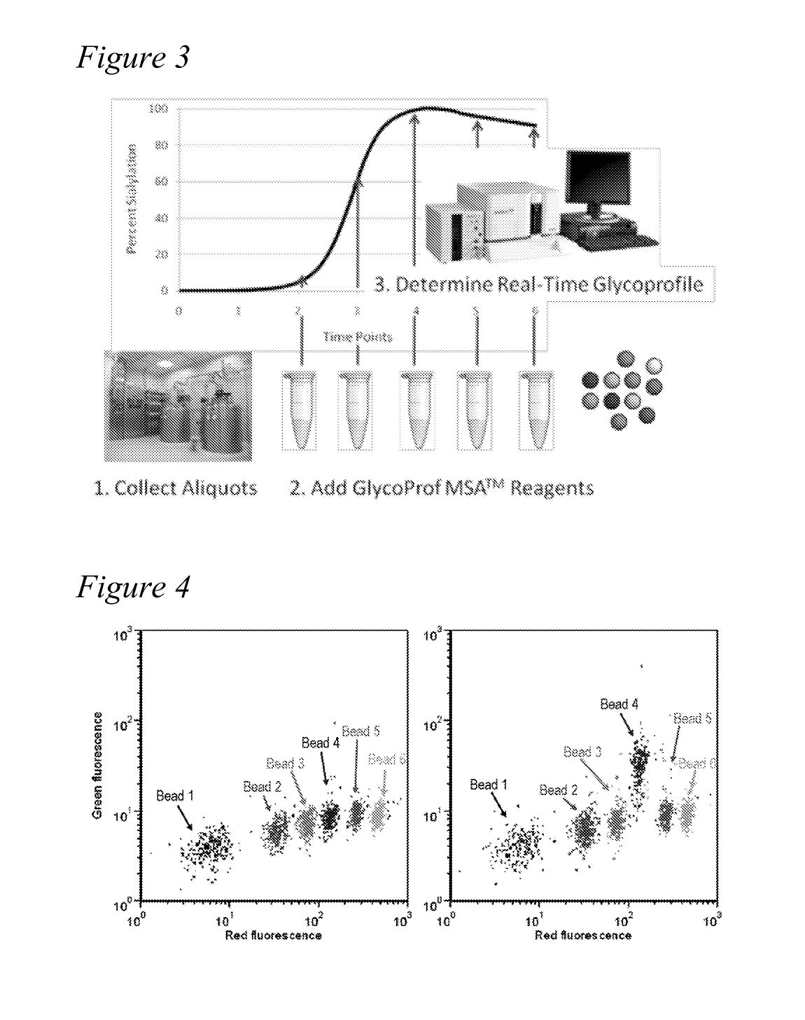

Glycoprofiling during Glycoprotein Expression. The process for in-process glycoprofiling is presented in FIG. 3. In order to maximize the turnaround time for this application, a secondary reagent, such as a labeled antibody or antibody fragment that is specific for the target glycoprotein, is employed for detection, eliminating the need to isolate and purify the expressed glycoprotein. N-Glycanase-PLUS (Prozyme) will be used to deglycosylate the glycoprotein specific antibody, prior to employing it in the assay to avoid interference. As it is not necessary to quantify the glycoprotein levels in order to determine the point at which the glycosylation profile reaches optimal levels, the use of a calibration curve, while possible, is not required.

### Example 5

### Confirmation

The accuracy of the glycoprofiles determined using the Glycoprofiling Multiplex Suspension Array method described herein will be confirmed by assaying glycoprotein samples whose glycoprofiles have already been determined or will be determined independently by complementary methods. Further, the glycoprofiles of biomedically relevant glycoproteins will be determined.

### Example 6

### Glycosidase Treatment

The Glycoprofiling Multiplexed Suspension Array described herein will be used to assay the effect on glycoprotein glycosylation profiles arising from glycosidase treatment with at least three glycosidases. Glycoprotein standards will be treated with glycosidases to generate altered glycosylation states, enabling an assessment of the sensitivity and accuracy of the Glycoprofiling Multiplexed Suspension Array when applied to glycoprotein samples. The necessary glycosidases are readily available and are routinely employed for glycan re-modeling. The glycosidases may be employed sequentially, for example to remove any terminal sialic acid, then to remove the subsequently-exposed Gal residues, then to remove the subsequently-exposed GlcNAc, etc. These will be applied to commercially available glycoproteins, such as RNase B, fetuin, sialoglycoprotein, glycophorin, etc. that present varying ratios of protein to glycan.

### Example 7

### Further Characterization

To establish standards for confirming batch consistency in the MSA reagents, lectins will be coupled to beads using standard protocols. The amount of unbound lectin will be measured by UV absorption. Additionally, the standardized glycans (Table 1) will be titrated against the beads to determine if the maximum loading capacity is within an acceptable range.

To quantify the ability of MSA reagents employed in a multiplexed analysis to reproduce the relative levels of stoichiometric mixtures of representative glycan structures, stoichiometric mixtures of the standardized glycans will be used to establish normalized fluorescence intensities. The maximum fluorescence intensity for each batch of glycoprofiling reagent beads will be determined by titrating with standardized glycans, such as those presented in Table 1. The glycan concentration at saturation will be employed to determine mixture stoichiometry. Based on this analysis the precision with which the Glycoprofiling MSA can reproduce the known glycan ratios will be determined.

For further testing, a Glycoprofiling MSA with specificity for at least 6 representative glycan structures associated with eukaryotic glycosylation, based on at least 12 glycan-binding reagents, will be extended by including reagents with additional and redundant specificities: such as the cholera toxin B subunit (CTB), as well as lectins from Canavalia ensiformis (ConA), Lens culinaris (LCH), Galanthus nivalis (GNA), peanut (PNA), Erythrina cristagalli (ECA), Phaseolus vulgaris (PHA), wheat germ (WGA), Sambucus nigra I (SNA-I), Maackia amurensis II (MAL II), Aleuria aurantia (AAL), Ulex europaeus (UEA), Polyporus squamosus (PSL), Griffonia simplicifolia II (GS II). Any of the wide variety of commercially available lectins and carbohydrate-binding antibodies, including, but not limited to, any of those described herein, may be used. In addition, engineered carbohydrate-binding proteins may be employed.

Reagents for incorporation into a glycoprofiling MSA will be selected that have specificity for at least six of the following glycosylation sequences: Neu5Acα2-6Gal, Neu5Acα2-3Galβ, terminal Galβ1-4GlcNAc, terminal GlcNAcβ, bisecting GlcNAcβ, terminal Manα, and terminal Fucα. In addition, wherever possible, selected reagents will have had their glycan binding patterns determined from specificity data generated by screening against over 300 glycans as reported by the Consortium for Functional Glycomics (CFG) (see the world wide web at functionalglycomics.org).

In addition to demonstrating the capabilities of the Glycoprofiling MSA method described herein to distinguish between standardized samples of glycans relevant to protein glycosylation patterns and to characterize glycosylation profiles with standardized samples of glycoproteins, the Glycoprofiling MSA method described herein will be used to monitor glycosylation profiles during bioprocessing. The glycosylation pattern of glycoproteins isolated at various time points during glycoprotein expression will be determined. Glycosylation profiles for purified glycoprotein samples typical of those in biopharmaceutical or research laboratory environments will be determined. The accuracy of the data obtained will be independently confirmed using complementary analytical methods. The performance of the glycoprofiling MSA products, kits, and method described herein will be evaluated in commercially available flow cytometer systems from at least three established vendors.

### Example 8

### Lectin MSA Reagents

As additional MSA glycoprofiling reagents, lectins, including, but are not limited to, any of those listed below, will be coupled to beads using standard protocols.

| Lectin | Specificity |
|---|---|
| Concanavalin A from Canavalia | α-Man; α-Glc (to a lesser extent); α-GlcNAc; α- |
| ensiformis (Jack bean) (Con A) | linked mannose; and succinyl Con A: α-Man, α-Glc |
| Datura stramonium (DSA) | β-GlcNAc,4GlcNAc oligomers; LacNAc; (β-1,4) |
| | linked N-acetylglucosamine oligomers, preferring |
| | chitobiose or chitotriose over a single N- |
| | acetylglucosamine residue, N-acetyllactosamine and |
| | oligomers containing repeating N-acetyllactosamine |
| | sequences |
| Dolichos biflorus agglutinin (DBA) | Terminal α-GalNAc; Blood Group A |
| Garden pea, Pisum sativum agglutinin | α-Man; α-Glc; α-GlcNAc; Biantennary and |
| (PSA) | triantennary oligosaccharides with core fucose; |
| | Fucα1,6-GlcNAc important in recognition; α-linked |
| | mannose-containing oligosaccharides, with an N- |
| | acetylchitobiose-linked α-fucose residue included in |
| | the receptor sequence |
| Jacalin, Artocarpus integrifolia | α-Gal; α-GalNAc; Core β1,3GalNAc (T Antigen); |
| | α-Gal-OMe; O-glycosidically linked |
| | oligosaccharides, preferring the structure galactosyl |
| | (β-1,3) N-acetylgalactosamine. will bind this |
| | structure even in a mono- or disialylated form |
| Lentil, Lens culinaris agglutinin (LCA | α-Man; α-Glc; α-GlcNAc; fucose linked to |
| or LcH) | chitobiose core of N-linked oligosaccharide |
| | enhances binding; α-linked mannose residues, by |
| | recognizing additional sugars as part of the receptor |
| | structure LCA has a narrower specificity than Con |
| | A. For example, an α-linked fucose residue attached |
| | to the N-acetylchitobiose portion of the core |
| | oligosaccharide markedly enhances affinity |
| Lotus, Lotus tetragonolobus lectin, | α-Fuc; alpha-linked L-fucose containing |
| winged or asparagus pea (LTL) | oligosaccharides; α-L-Fuc |
| Maackia amurensis (MAA) Lectin I | Neu5Acα2,3Galβ1,4GlcNAc; Sialic Acid; α-Neu |
| (MAL I) and Lectin II (MAL II) | NAc (2→3)Gal; MAL I: galactosyl (β-1,4) N- |
| | acetylglucosamine structures. Maackia amurensis |
| | lectin I seems to tolerate substitution of N- |
| | acetyllactosamine with sialic acid at the 3 position |
| | of galactose however, MAL I does not appear to |
| | bind this structure when substitution with sialic acid |
| | is on the 6 position of galactose; MAL II: appears to |
| | bind sialic acid in an (α-2,3) linkage |
| Peanut, Arachis hypogaea (PNA) | β-Gal; β-Gal(1→3)GalNAc; Galβ1,3GalNAc (T |
| | antigen); Galβ1,3GalNAcα-O—Me (T antigen, α- |
| | Methyl Glycoside); galactosyl (β-1,3) N- |
| | acetylgalactosamine |
| Red kidney bean, Phaseolus vulgaris | α-GalNAc; β-GalNAc; Complex biantennary |
| Erythroagglutinin (PHA-E) | oligosaccharides with outer galactose and bisecting |
| | GlcNAc |
| Red kidney bean, Phaseolus vulgaris | α-GalNAc; β-GalNAc; Triantennary and |
| Leucoagglutinin (PHA-L) | tetraantennary complex oligosaccharides |
| Potato, Solanum tuberosum (STA) | β-GlcNAc; GlcNAcβ1,4-R; oligomers of N- |
| | acetylglucosamine and some bacterial cell wall |
| | oligosaccharides containing N-acetylglucosamine |
| | and N-acetylmuramic acid |
| Sambucus nigra (SNA or EBL) | Neu5Acα2,6Gal; Neu5Acα2,6GalNAc; β-Gal; |
| | Sialic Acid; α-NeuNAc(2→6) Gal/GalNAc; sialic |
| | acid attached to terminal galactose in (α-2,6), and to |
| | a lesser degree, (α-2,3), linkage |
| Slug, Limax flavus (LFA) | Neu5Ac; NeuGc; Sialic Acid |
| Soybean, Glycine soja or Glycine max | α- or β-GalNAc; GalNAc; Gal (to a lesser extent); |
| (SBA) | oligosaccharide structures with terminal α- or β- |
| | linked N-acetylgalactosamine, and to a lesser extent, |
| | galactose residues |
| Tomato, Lycopersicon esculentum | GlcNAcβ1,4GlcNAc oligomers; β-GlcNAc; N- |
| (LEA or LEL or TL) | acetylglucosamine oligomers, tomato lectin prefers |
| | trimers and tetramers of this sugar |
| Tritrichomonas mobilensis | Neu5Ac; NeuGc (to a lesser extent) |
| Ulex europaeus I (UEA I) | Blood Group H oligosaccharides, |
| | Fucα1,2Galβ1,4GlcNAc; α-Fucose; α-linked fucose |
| | residues; α-L-Fuc |
| Vicia villosa (VVA or VVL) | Tn antigen; GalNAcα1-O-Serine; mannose; α-Man? |
| | β-Man?; α-GalNAc; alpha- or beta-linked terminal |
| | N-acetylgalactosamine, especially a single alpha N- |
| | acetylgalactosamine residue linked to serine or |
| | threonine in a polypeptide (the “Tn antigen”) |
| Wheat Germ agglutinin, Triticum | (GlcNAc)2; (GlcNAc)3; Neu5Ac; β-GlcNAc; Sialic |
| vulgaris (WGA) | Acid; NeuNAc; N-acetylglucosamine, with |
| | preferential binding to dimers and trimers of this |
| | sugar. WGA can bind oligosaccharides containing |
| | terminal N-acetylglucosamine or chitobiose; |
| | succinylated WGA does not bind to sialic acid |
| | residues, unlike the native form, but retains its |
| | specificity toward N-acetylglucosamine |
| Wisteria floribunda (WFA or WFL) | Terminal GalNAcβ1,4- >> Terminal GalNAcα1,3- |
| | or Terminal GalNAcβ1,3-; α-GalNAc; β-GalNAc; |
| | GalNAc; carbohydrate structures terminating in N- |
| | acetylgalactosamine linked alpha or beta to the 3 or |
| | 6 position of galactose |
| Galanthus nivalis (GNA or GNL) | α-Man; non-reduc. D-Man; (α-1,3) mannose |
| | residues; will not bind alpha linked glucose; |
| Vicia faba (VFA) | α-Man; α-Glc; α-GlcNAc; |
| Narcissus pseudonarcissus (NPA or | α-Man? β-Man?; alpha linked mannose, preferring |
| NPL) | polymannose structures containing (α-1,6) linkages |
| Chick pea, Cicer arietinum (CPA) | α-Man? β-Man?; Fetuin |
| Griffonia (Bandeiraea) simplicifolia II | α-GlcNAc; β-GlcNAc; alpha- or beta-linked N- |
| (GS II or GSL II) | acetylglucosamine residues, increasing the number |
| | of N-acetylglucosamine residues beyond two does |
| | not improve affinity; recognize exclusively alpha- |
| | or beta-linked N-acetylglucosamine residues on the |
| | nonreducing terminal of oligosaccharides |
| Laburnum alpinum (LAA) | β-GlcNAc |
| Oryza sativa (OSA) | β-GlcNAc |
| Ulex europaeus II (UEA II) | β-GlcNAc |
| Urtica dioica (UDA) | β-GlcNAc |
| Vigna radiate (VRA) | α-Gal |
| Psophocarpus tetragonolobus, Winged | α-Gal? β-Gal; GalNAc, Gal; PTL I: alpha linked |
| bean (PTA) Lectin I (PTL I) or Lectin | galactosamine; PTL II: binds preferentially to |
| II (PTL II) | galactosides, with N-acetylgalactosamine being the |
| | most inhibitory monosaccharide. However, in |
| | contrast to PTL I, this lectin prefers the beta |
| | anomeric configuration. PTL II shows a high |
| | affinity toward blood group H structures and the T- |
| | antigen |
| Garden snail, Helix aspersa (HAA) | α-GlcNAc; α-GalNAc; GalNAc |
| Griffonia (Bandeiraea) simplicifolia I | α-Gal; α-GalNAc; mixture of the five isolectins: A- |
| (GS I or BS I or GSL I) | rich lectin specific for α-N-acetylgalactosamine |
| | residues, while the B-rich lectin specific for α- |
| | galactose residues; Isolectin B4 (GS I-B4 or BS I- |
| | B4): α-Gal; Isolectin A4 (GS I-A4 or Bs I-A4): α- |
| | GalNAc |
| Edible snail, Helix pomatia (HPA) | α-GalNAc; GalNAc |
| Maclura pomifera (MPA or MPL) | α-Gal; α-GalNAc; alpha linked N- |
| | acetylgalactosamine structures |
| Colchicum autumnale (CA) | α-Gal? β-Gal? α-GalNAc? B-GalNAc? |
| mistletoe, Viscum album (VAA) | β-Gal |
| Allomyrina dochotoma (Allo A) | β-Gal |
| mushroom, Agaricus bisporus (ABA) | β-Gal; β-Gal(1→3)GalNac |
| Abrus precatorius (APA) | β-Gal |
| Cytisus scoparius (CSA) | β-Gal |
| Trichosanthes kirilowii (TKA) | β-Gal |
| castor bean, Ricinus communis I | β-Gal; oligosaccharides ending in galactose but may |
| (RCA I); RCA₁₂₀ | also interact with N-acetylgalactosamine |
| castor bean, Ricinus communis II | β-Gal; β-GalNAc; galactose or N- |
| (RCA II); RCA₆₀, Ricin, A chain | acetylgalactosamine residues |
| coral tree, Erythrina cristagalli (ECA | α-Gal; β-Gal; α-GalNAc; β-GalNAc; β- |
| or ECL) | Gal(1→4)GlcNAc; galactose residues and appears |
| | to have the highest binding activity toward |
| | galactosyl (β-1,4) N-acetylglucosamine |
| Siberian pea tree, Caragana | α-Gal; β-Gal; α-GalNAc; β-GalNAc; GalNAc |
| arborescens (CAA) | |
| Phaseolus lunatus (LBA) | α-GalNAc |
| Bauhinia purpurea (BPA or BPL) | α-GalNAc; β-GalNAc; galactosyl (β-1,3) N- |
| | acetylgalactosamine structures but oligosaccharides |
| | with a terminal alpha linked N-acetylgalactosamine |
| | can also bind |
| Aegopodium podagraria (APP) | α-GalNAc; β-GalNAc |
| Bryonia dioica (BDA) | α-GalNAc; β-GalNAc |
| Tulip lectin (TL) | α-GalNAc; β-GalNAc |
| Sophora japonica (SJA) | β-GalNAc; carbohydrate structures terminating in |
| | N-acetylgalactosamine and galactose residues, with |
| | preferential binding to β anomers |
| Anguilla Anguilla (AAA) | α-Fucose |
| horseshoe crab, Limulus polyphemus | Sialic Acid; NeuNAc; (Neu5Ac)•2,6- |
| (LPA) | GalNAc group |
| Homarus americanus (HMA) | α-GalNAc; α-Fucose; Sialic Acid |
| Cancer antennarius (CCA) | Sialic Acid |
| Vicia graminea (VGA) | |
| Euonymus europaeus (EEL) | type 1 or type 2 chain blood group B structures but |
| | will bind other oligosaccharides containing |
| | galactosyl (α-1,3) galactose; type 1 chain blood |
| | group H structures; |
| Robinia pseudoaccacia (RPA) | |
| Salvia horminum (SHA) | |
| Salvia sclarea (SSA) | |
| Perseau Americana (PAA) | |
| Mangifera indica (MIA) | |
| Iberis amara (IAA) | |
| Sarothamnus scoparius (SRA) | |
| Trifolium repens (RTA) | |
| Green marine algae, Codium fragile | GalNAc |
| Human Galectin-1 (Gal-1) | β-Gal |
| Human Galectin-3 (Gal-3) | β-Gal |
| Human Galectin-3C | β-Gal |
| red kidney bean, Phaseolus Vulgaris | |
| Agglutinin (PHA-E + L) | |
| red kidney bean, Phaseolus vulgaris | |
| Phytohemagglutinin (PHA-P) | |
| red kidney bean, Phaseolus vulgaris | |
| Mucoprotein (PHA-M) | |
| Pokeweed, Phytolacca americana | (GlcNAc)3 |
| (PWM) | |
| Pseudomonas aeruginosa (PA-I) | Gal |
| Rat Galectin-8 (Gal-8) | β-Gal |
| Aleuria Aurantia Lectin (AAL) | fucose linked (α-1,6) to N-acetylglucosamine or to |
| | fucose linked (α-1,3) to N-acetyllactosamine related |
| | structures |
| Amaranthus Caudatus Lectin (ACL or | galactosyl (β-1,3) N-acetylgalactosamine structure |
| ACA) | (“T-antigen”), tolerate sialic acid substitution at the |
| | 3 position of galactose in the “T” antigen |
| Hippeastrum Hybrid Lectin (HHL or | only alpha mannose residues, not alpha glucosyl |
| AL) | structures. an extended binding site for |
| | polymannose structures, not requiring mannose to |
| | be at the non-reducing terminus. binds both (α-1,3) |
| | and (α-1,6) linked mannose structures, as well as |
| | some yeast galactomannans |
| Ricin B Chain | |

### Example 9

### Anticarbohydrate Antibody MSA Reagents

As additional MSA glycoprofiling reagents, anti-carboydrate antibodies will be coupled to beads using standard protocols. For example, antibody-bearing beads may be prepared by incubating 20 μL of carboxylated microspheres (5-7.2×10⁷/mL) with 20 μL antibody (1 mg/mL) in PBS for 15 min. Two microliters of NHS (50 mg/mL) and 2 μL of EDAC (50 mg/mL) were added, and the beads incubated for one hour at 4° C. Microspheres are washed twice with PBS plus 0.02% Tween20 (PBST) and resuspended to a concentration of 5×10⁷/mL.

Anti-carbohydrate antibodies include, but are not limited to, any of the following. Blood Group H n/ab antigen (86-M) Antibody (Abcam No. ab24776; Santa Cruz Biotechnology No. sc-52372); Blood Group A antigen (9A) Antibody (Abcam No. ab20131; GeneTx No. GTX40131; Santa Cruz Biotechnology No. sc-53180); Blood Group A antigen (HE-193) Antibody (Abcam No. ab2521; GeneTx No. GTX22521; Santa Cruz Biotechnology No. sc-59460); Blood Group A antigen (HE-195) Antibody (Abcam No. ab2522; GeneTx No. GTX22522); Blood Group A antigen (T36) Antibody (Abcam No. ab3353; GeneTx No. GTX23353); Blood Group A, B and H antigens (HE-10) Antibody (Abcam No. ab2523; GeneTx No. GTX22523; Santa Cruz Biotechnology No. sc-59459); Blood Group A1B antigen (HE-24) Antibody (Abcam No. ab2525; GeneTx No. GTX22525); Blood Group AB antigen (Z5H-2/Z2A) Antibody (Abcam No. ab24223); Blood Group antigen Precursor (K21) Antibody (Abcam No. ab3352; GeneTx No. GTX23352); Blood Group B antigen (CLCP-19B) Antibody (Abcam No. ab3354); Blood Group B antigen (HEB-29) Antibody (Abcam No. ab2524; GeneTx No. GTX22524; Santa Cruz Biotechnology No. sc-59463); Blood Group B antigen (Z5H-2) Antibody (Abcam No. ab24224); Blood Group H ab antigen (87-N) Antibody (Abcam No. ab24222; Santa Cruz Biotechnology No. sc-52369); Blood Group A1, A2 antigen (87-G) Antibody (Santa Cruz Biotechnology No. sc-52368); Blood Group H1 (O) antigen (17-206) Antibody (Abcam No. ab3355; GeneTx No. GTX23355); Blood Group H1+ Blood Group H2 (0.BG.5) Antibody (Abcam No. ab31754); Blood Group H2 (0.BG.6) Antibody (Santa Cruz Biotechnology No. sc-59466); Blood Group Kell antigen (0.BG.7) Antibody (Abcam No. ab31771); Blood Group H2 antigen (BRIC231) Antibody (Abcam No. ab33404); Blood Group Kell Antigen (BRIC 203) Antibody (Abcam No. ab11463); Sialyl Tn (BRIC111) Antibody (Abcam No. ab24005); Blood Group Wrb (BRIC14) Antibody (Santa Cruz Biotechnology No. sc-59476); Blood Group H2 (BRIC231) Antibody (Santa Cruz Biotechnology No. sc-59467); Blood Group Kell antigen (MM0435-12X3) Antibody (Abcam No. ab90456); CD239 (MM0107-1M39) Antibody (Abcam No. ab89142); Blood Group Kell Antigen (RM0118-7L32) Antibody (Abcam No. ab86793); Blood Group Lewis (2Q398) Antibody (Abcam No. ab68390); Blood Group Lewis a (7LE) Antibody (Abcam No. ab3967; GeneTx No. GTX23967; Santa Cruz Biotechnology No. sc-51512); Blood Group Lewis a (PR 5C5) Antibody (Abcam No. ab70473); Blood Group Lewis a (PR 4D2) Antibody (Santa Cruz Biotechnology No. sc-53181); Blood Group Lewis a (SPM522) Antibody (Abcam No. ab64099; Santa Cruz Biotechnology No. sc-135725); CA19-9 (SPM110) Antibody (Abcam No. ab15146; Santa Cruz Biotechnology No. sc-56506); Blood Group Lewis a (SPM279) Antibody (Santa Cruz Biotechnology No. sc-52988); Blood Group Lewis a (T174) Antibody (Abcam No. ab3356; GeneTx No. GTX23356; Santa Cruz Biotechnology No. sc-59469); Blood Group Lewis b (2-25LE) Antibody (Abcam No. ab3968; GeneTx No. GTX23968; Santa Cruz Biotechnology No. sc-51513); Blood Group Lewis b antibody (LWB01; same as 2-25LE) Antibody (Abcam No. ab44959; GeneTx No. GTX72378); Blood Group Lewis b (T218) Antibody (Abcam No. ab3357; Santa Cruz Biotechnology No. sc-59470); Blood Group Lewis x (4C9) Antibody (Abcam No. ab52321; Santa Cruz Biotechnology No. sc-69905); Blood Group Lewis x (P12) Antibody (Abcam No. ab3358; GeneTx No. GTX23358; Santa Cruz Biotechnology No. sc-59471); Blood Group Lewis y (A70-C/C8) Antibody (Abcam No. ab23911; Santa Cruz Biotechnology No. sc-59472); Blood Group Lewis y (F3) antibody (Abcam No. ab3359; GeneTx No. GTX23359); Blood Group N antigen (DRF-8) Antibody (Abcam No. ab24217; Santa Cruz Biotechnology No. sc-52374); Blood Group Tn antigen (Tn 218) Antibody (Abcam No. ab76752); Blood Group Wrb (E6) Antibody (Abcam No. ab50293; Santa Cruz Biotechnology No. sc-81763); Blood group H inhibitor (97-I) Antibody (Abcam No. ab24213); CA19-9 (0.N.36) Antibody (Abcam No. ab33181); CA19-9 (121SLE) Antibody (Abcam No. ab3982); Sialyl Lewis a (121SLE) Antibody (Santa Cruz Biotechnology No. sc-51696); CA19-9 (BC/121SLE) Antibody (Abcam No. ab2707); CA19-9 (192) Antibody (Abcam No. ab25802; Santa Cruz Biotechnology No. sc-59480); CA19-9 (241) Antibody (Santa Cruz Biotechnology No. sc-59481); CA19-9 (8.F.26) Antibody (Santa Cruz Biotechnology No. sc-73411); CD77 (38-13) Antibody (Abcam No. ab19795); Blood Group M antigen (GH-9) Antibody (Abcam No. ab24215; Santa Cruz Biotechnology No. sc-52373); Sialyl Tn (STn 219) Antibody (Abcam No. ab76754); CD15 (28) Antibody (Abcam No. ab20137); CD15 (DU-HL60-3) Antibody (Abcam No. ab13453); CD15 murine monoclonal (MC480) (Abcam No. ab16285); CD15 (MY-1) Antibody (Abcam No. ab754); Blood Group B antigen (Z5H-2) Antibody (GeneTx No. GTX44224; Santa Cruz Biotechnology No. sc-69952); Blood Group AB antigen (Z5H-2/Z2A) Antibody (GeneTx No. GTX44223; Santa Cruz Biotechnology No. sc-52370); Blood Group Lewis a/b (HEA164) Antibody (Santa Cruz Biotechnology No. sc-73368); Blood Group Lewis a (B369) Antibody (Santa Cruz Biotechnology No. sc-59468); Blood Group A antigen (Z2A) Antibody (Santa Cruz Biotechnology No. sc-69951); Blood Group A antigen (B45.1) Antibody (Santa Cruz Biotechnology No. sc-59457); Blood Group A antigen (B480) Antibody (Santa Cruz Biotechnology No. sc-59458); Blood Group A1, A2, A3 antigen (1V015) Antibody (Santa Cruz Biotechnology No. sc-70427); Blood Group A1, A2, A3 antigen (Z2B-1) Antibody (Santa Cruz Biotechnology No. sc-52367); Blood Group B antigen (89-F) Antibody (Santa Cruz Biotechnology No. sc-52371); Blood Group H2 (A46-B/B10) Antibody (Santa Cruz Biotechnology No. sc-65680); Blood Group H2 (A51-B/A6) Antibody (Santa Cruz Biotechnology No. sc-65682); Blood Group M antigen (1.B.710) Antibody (Santa Cruz Biotechnology No. sc-70428); Forssman Antigen (M1/87) Antibody (Santa Cruz Biotechnology No. sc-23939); Forssman Antigen (M1/87.27.7.HLK) Antibody (Santa Cruz Biotechnology No. sc-81724); CD15s (CHO131) Antibody (Santa Cruz Biotechnology No. sc-32243); and CD15s (5F18) Antibody (Santa Cruz Biotechnology No. sc-70545).

### Example 10

### Disease Targets

The glycoprofiling MSA technology described herein may be applied to the diagnosis of a variety of diseases, including, but not limited to, any of those described below.

| Target | Disease | Current Reagent |
|---|---|---|
| beta(1,6)-branching | Breast Carcinoma: During the oncogenesis of breast | PHA-L |
| of polylactosamine | carcinoma, the glycosyltransferase known as N- | (Kaneda et al., |
| chains | acetylglucosaminyltransferase Va (GnT-Va) | 2002, J Biol |
| GlcNAcb(1-6)Gal | transcript levels and activity are increased due to | Chem; 277: 16928-16935) |
| mostly endo | activated oncogenic signaling pathways. Elevated | |
| | GnT-V levels leads to increased β(1,6)-branched N- | |
| | linked glycan structures on glycoproteins | |
| | (Abbott et al., 2008, J Proteome Res; 7(4): 1470-80) | |
| Polylactosamine | Cold Agglutinin Disease: Auto-antibodies react with | DSL, DSA |
| basis for beta(1,6) | the “i” antigen, can be triggered by infection with M. pneumonia. | |
| branching of “i” | | |
| Blood Group | | |
| Antigen | | |
| [Galb(1- | | |
| 4)GlcNAcb(1-3)]n | | |
| Polylactosamine | Cold Agglutinin Disease | DSL, DSA |
| basis for beta(1,6) | | |
| branching of “i” | | |
| Blood Group | | |
| Antigen | | |
| [GlcNAcb(1- | | |
| 3)Galb(1-4)]n | | |
| Bisecting GlcNAc | Related to antibody effector function, autoimmune | PHA-E |
| GlcNAcb(1-4)Man | disease, antigen binding | (Kaneda et al., |
| | (Arnold et al., 2007, Ann Rev Immunol; 25: 21-50) | 2002, J Biol |
| | | Chem; 277: 16928-16935) |
| Bisecting GlcNAc | Normal liver cells and primary adult hepatocytes are | PHA-E |
| GlcNAcb(1-4)Man | characterized by a very low level of GlcNAc- | (Kaneda et al., |
| | transferase-III activity, whereas human hepatoma | 2002, J Biol |
| | cells exhibited high activities | Chem; 277: 16928-16935) |
| | (Song et al., 2001, Cancer Invest; 19(8): 799-807) | |
| core alpha-1,6- | Hepatocellular carcinoma: woodchucks diagnosed | Array of |
| linked fucose | with HCC have dramatically higher levels of serum- | lectins from Lens |
| Fuca(1-6)GlcNAcb | associated core α-1,6-linked fucose, as compared with | culinaris, Pisum |
| | woodchucks without a diagnosis of HCC | sativum, |
| | (Block et al., 2005, Proc Natl Acad Sci USA; | and Vicia faba. |
| | 102: 779-84) | |
| core alpha-1,6- | Related to antibody effector function, autoimmune | Array of |
| linked fucose | disease, antigen binding | lectins from Lens |
| Fuca(1-6)GlcNAcb | (Arnold et al., 2007, Ann Rev Immunol; 25: 21-50) | culinaris, Pisum |
| | | sativum, |
| | | and Vicia faba. |
| Outer arm | Pancreatic Cancer: Forty-four oligosaccharides were | ConA, UEA-I |
| fucosylation | found to be distinct in the pancreatic cancer serum. | (ConA lectin |
| Fuca(1-2)Gal (A, | Increased branching of N-linked oligosaccharides and | affinity |
| B, H, Le^{y}, Le^{b} | increased fucosylation and sialylation were observed | chromatography, |
| antigen) | in samples from patients with pancreatic cancer | the recovery for |
| | (Zhao et al., 2007, J Proteome Res; 6(3): 1126-1138) | N-linked glycan |
| | | structures with a |
| | | mannose core |
| | | such as complex |
| | | type glycans is |
| | | lower than the |
| | | high mannose |
| | | glycan structure |
| | | proteins.) |
| Fuca(1-2)Galb | Prostate and Colon Cancer: A characteristic feature | PNA |
| | of tumor progression in distal colon and rectum is the | |
| | expression of the blood group determinants Le^{b}, H- | |
| | type 2 and Le^{y}, as well as the glycolipid Globo H, | |
| | which contain the motif Fuca(1-2)Galβ-R | |
| | (Chandrasekaran et al., 2002, Glycobiol; 12: 153-162) | |
| Outer arm | (Chandrasekaran et al., 2002, Glycobiol; 12: 153-162) | Blood Group |
| fucosylation | | Lewis x antibody |
| Fuca(1-3)GlcNAc | | [P12] |
| (Le^{x} antigen) | | |
| Antigen Le^{y} | Aberrant glycosylation has been associated with the | MAb AH6 |
| Fuca(1-2)Galb(1- | malignant phenotype in various tissues, and certain | MAb B3 |
| 4)[Fuca(1- | alterations in oligosaccharides have been associated | Antibody AH6, |
| 3)]GlcNAcb1-R | with the metastatic process and poor patient survival | IgM and TKH2, |
| | in several carcinomas. These include increase in | IgG. |
| | Lewis y (Le^{y}), Sialyl Lewis x (Sle^{x}), Sialyl Tn (STn), | |
| | and Tn expression (Davidson et al., 2000, Hum | |
| | Pathol; 31: 1081-1087). | |
| Le^{x} epitope | Cancer Metastasis: N-linked glycosylation from a | All by MS |
| Galb(1-4)[Fuca(1- | nonmetastatic brain tumor cell line and two different | |
| 3)]GlcNAcb(1- | metastatic brain tumor cells were compared (Prien et | |
| 3)Gal | al., 2008, Glycobiol; 18: 353-366) | |
| Outer arm | (Prien et al., 2008, Glycobiol; 18: 353-366) | Blood Group |
| fucosylation | | Lewis a antibody |
| Fuca(1-4)GlcNAc | | [SPM522] |
| (Le^{a}, Le^{b} antigen) | | |
| terminal Neu5Ac | Related to antibody effector function, autoimmune | SNA |
| Neu5Aca(2-6)Gal | disease, antigen binding (Arnold et al., 2007, Ann Rev | |
| | Immunol; 25: 21-50) | |
| Terminal Neu5Ac | Pancreatic Cancer: Forty-four oligosaccharides were | ConA |
| Neu5Aca(2-6)Gal | found to be distinct in the pancreatic cancer serum. | (lectin affinity |
| | Increased branching of N-linked oligosaccharides and | chromatography, |
| | increased fucosylation and sialylation observed in | the recovery for |
| | samples from patients with pancreatic cancer | N-linked glycan |
| | (Zhao et al., 2007, J Proteome Res; 6: 1126-1138) | structures with a |
| | | mannose core |
| | | such as complex |
| | | type glycans is |
| | | lower than the |
| | | high mannose |
| | | glycan structure |
| | | proteins.) |
| Terminal Neu5Ac | (Zhao et al., 2007, J Proteome Res; 6: 1126-1138) | MAA |
| Neu5Aca(2-3)Gal | | |
| Sialyl-Lewis X | (Zhao et al., 2007, J Proteome Res; 6: 1126-1138) | MAb 2H5 |
| Neu5Aca(2- | | Antibody 2H5, |
| 3)Galb(1-4)[Fuca1- | | IgM (PharMingen, |
| 3]GlcNAc-R | | Becton Dickinson, |
| | | San Jose, CA) |
| Neu5Aca(2-3)Gal | Influenza receptor (Horimoto and Kawaoka, 2005, | MAA |
| | Nat Rev Microbiol; 3: 591-600) | |
| Neu5Aca(2-6)Gal | Influenza receptor (Horimoto and Kawaoka, 2005, | LPA, SNA |
| | Nat Rev Microbiol; 3: 591-600) | |
| terminal Neu5Ac | IgA nephropathy: the IgA glycoform from IgAN | MAA |
| Neu5Aca(2-3)Gal | patients highly expressing GalNAc or Neu5Ac- | |
| | 2,6,GalNAc significantly depressed the Mesangial | |
| | Cell proliferation rate (Coppo and Amore, 2004, | |
| | Kidney International; 65: 1544-1547) | |
| terminal Neu5Ac | IgA nephropathy: N-linked (Coppo and Amore, 2004, | SNA |
| Neu5Aca(2- | Kidney International; 65: 1544-1547) | |
| 6)GalNAc | | |
| Sialyl-Tn | Common feature in mucins associated with | MAb TKH2 |
| Neu5Aca(2- | carcinomas | |
| 6)GalNAca1-O- | | |
| Ser/Thr | | |
| Found on MUC1 | | |
| terminal Gal | IgA nephropathy: N-linked (Coppo and Amore, 2004, | WGA, Jacalin |
| Galb1-3GalNAc | Kidney International; 65: 1544-1547) | |
| TF-antigen | Associated with carcinomas (colon cancer): The | PNA, ABA |
| Galb(1-3)GalNAc | glycosylation changes include increased expression of | |
| found on MUC1 | onco-fetal carbohydrates, such as the galactose- | |
| | terminated Thomsen-Friedenreich antigen | |
| | (Galβ1,3GalNAcα-), increased sialylation of terminal | |
| | structures and reduced sulphation | |
| terminal | Related to antibody effector function, autoimmune | MAL I |
| galactosylation | disease, antigen binding (Arnold et al., 2007, Ann Rev | |
| Galb(1-4)GlcNAc | Immunol; 25: 21-50) | |
| Terminal GalNAc | IgA nephropathy: | SBA |
| GalNAc-OSer/Thr | (Amore and Coppo, 2000 Nephron 86: 255-259) | |
| Tn Antigen | Common feature in mucins associated with | MAb HB-Tn1 |
| GalNAca-O- | carcinomas | Antibody HB-Tn1, |
| Ser/Thr | | IgM (Dako, |
| Found on MUC1 | | Glostrup, |
| | | Denmark) VVL, |
| | | VVA |
| terminal GlcNAc | Related to antibody effector function, autoimmune | PHA-L |
| GlcNAcb(1-2)Man | disease, antigen binding (Arnold et al., 2007, Ann Rev | |
| | Immunol; 25: 21-50) | |
| terminal GlcNAc | (Arnold et al., 2007, Ann Rev Immunol; 25: 21-50) | STA |
| GlcNAcb(1-4)Man | | |
| terminal GlcNAc | (Arnold et al., 2007, Ann Rev Immunol; 25: 21-50) | |
| GlcNAcb(1-6)Man | | |
| N-glycolyl GM3 | This epitope is a molecular marker of certain tumor | MAb 14F7 |
| Neu5Gca(2-3)Gal | cells and not expressed in normal human tissues | |
| | (Arnold et al., 2007, Ann Rev Immunol; 25: 21-50) | |
| Terminal GlcNAc | Type II Diabetes: increased intracellular glycosylation | anti-O-GlcNAc |
| GlcNAcb-O- | of proteins via O-GlcNAc can induce insulin | antibody RL-2 and |
| Ser/Thr | resistance and that a rodent model with genetically | ERK-2, MAb |
| | elevated O-GlcNAc levels in muscle and fat displays | CTD110.6, |
| | hyperleptinemia (Lim et al., 2008, J Proteome Res; | |
| | 7(3): 1251-63; Comer et al., 2001, Anal Biochem; | |
| | 293: 169-177) | |
| tumor-associated | Tumor associated antigen: Antigen initially detected | The monoclonal |
| antigen 19-9 | in a human colorectal cell line | antibody CO 19-9 |
| Neu5Aca(2- | | is specific for the |
| 3)Galb(1- | | 19-9 |
| 3)[Fuca(1- | | antigen and does |
| 4)]GlcNAc | | not cross-react |
| Galb(1-3)GlcNAc | | with Le^{a} |
| Le^{a} blood group | Tumor associated antigen: | The monoclonal |
| antigen component, | (Bechtel et al., 1990, J Biol Chem; 265: 2028-2037) | antibody CO 19-9 |
| Galb(1-3)[Fuca(1- | | is specific for the |
| 4)]GlcNAc | | 19-9 |
| | | antigen and does |
| | | not cross-react |
| | | with Le^{a} |
| Globo H | Breast Cancer: The cell-surface glycosphingolipid | MBr1 (IgM, |
| Fuca(1-2)Galb(1- | Globo H is a member of a family of antigenic | Alexis |
| 3)GlcNAcb(1- | carbohydrates that are highly expressed on a range of | Biochemicals, |
| 3)Gala(1-4)Galb(1- | cancer cell lines, especially breast cancer cells | Lausen, |
| 4)Glcb | (Huang et al., 2006, Proc Natl Acad Sci USA; 103: 15-20; | Switzerland) and |
| | Wang et al., 2008, Proc Natl Acad Sci USA; | VK-9 (IgG). |
| | 105: 11661-11666) | |
| Globo H | Breast Cancer: | Glycoope antibody |
| Fuca(1-2)Galb(1- | (Huang et al., 2006, Proc Natl Acad Sci USA; 103: 15-20; | to Globo H A69- |
| 3)GlcNAcb(1- | Wang et al., 2008, Proc Natl Acad Sci USA; | A/E8 |
| 3)Gala(1-4)Galb(1- | 105: 11661-11666) | |
| 4)Glcb | | |
| Gb3 | The trisaccharide glycolipid Gb-3 is a receptor for | Anti-Gb3 Isotype |
| Gala(1-4)Glcb(1- | Shiga-like toxins and has recently been implicated in | IgM (1A4) |
| 4)Glcb-Cer | the entry of HIV-1 into cells (Werz et al., 2007, J Am | |
| | Chem Soc; 129: 2770-2771) | |
| Forssman Antigen | Various cancer tissues (Hakomori, 1984, Ann Rev | Forssman Antigen |
| GalNAca(1- | Immunol; 2: 103-26) | (M1/87) Antibody |
| 3)GalNAcb(1-3)- | | |
| Gala(1-4)Galb(1- | | |
| 4)Glcb(1- | | |
| Forssman Antigen | Various cancer tissues (Hakomori, 1984, Ann Rev | Forssman Antigen |
| GalNAca(1- | Immunol; 2: 103-26) | (M1/87) Antibody |
| 3)GalNAcb(1-3)- | | |
| Gala(1-4)Galb(1- | | |
| 4)Glcb(1- | | |
| GlcNAcb(1- | Common to all N-linked glycans and fundamental to | N/A |
| 4)GlcNAcb-N-Asn | many of the glycans in this table | |
| GlcNAcb(1- | Common to all N-linked glycans and fundamental to | N/A |
| 4)GlcNAcb-N-Asn | many of the glycans in this table | |
| GlcNAcb(1- | Hepatocellular carcinoma: woodchucks diagnosed | N/A |
| 4)[Fuca(1- | with HCC have dramatically higher levels of serum- | |
| 6)GlcNAcb-N-Asn | associated core α-1,6-linked fucose, as compared with | |
| | woodchucks without a diagnosis of HCC (Block et | |
| | al., 2005, Proc Natl Acad Sci USA; 102: 779-84) | |
| GlcNAcb(1- | Hepatocellular carcinoma: woodchucks diagnosed | N/A |
| 4)[Fuca(1- | with HCC have dramatically higher levels of serum- | |
| 6)GlcNAcb-N-Asn | associated core α-1,6-linked fucose, as compared with | |
| | woodchucks without a diagnosis of HCC (Block et | |
| | al., 2005, Proc Natl Acad Sci USA; 102: 779-84) | |
| [6S]GleNS- | Glycosaminoglycans include heparin and are | N/A |
| [2S]IdoA | associated with viral adhesion (herpes) and some | |
| | cancers | |
| GlcNS-IdoA | Glycosaminoglycans include heparin and are | N/A |
| | associated with viral adhesion (herpes) and some | |
| | cancers | |
| GlcNS-GlcA | Glycosaminoglycans include heparin and are | N/A |
| | associated with viral adhesion (herpes) and some | |
| | cancers | |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A composition comprising a plurality of individually addressable particles, each individually addressable particle comprising an external surface and having linked to said external surface a separate carbohydrate binding molecule.

2. The composition of claim 1, wherein the carbohydrate binding molecules are independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains, and aptamers.

3. The composition of claim 2, wherein the LECTENZ molecule is derived from an enzyme selected from the group consisting of a glycosidase enzyme, a glycosyltransferase enzyme, polysaccharide lyase enzyme, sulfatase enzyme, a sulfotransferase enzyme, a ligase enzyme, an amidase enzyme, and an epimerase enzyme.

4. The composition of claim 2, wherein the LECTENZ molecule is derived from PNGaseF or O-GlcNAcase.

5. The composition of claim 2, wherein the individually addressable particle comprises a bead or a nanoparticle.

6. The composition of claim 5, wherein each individually addressable particle is separately labeled with a detectable label.

7. The composition of claim 6, wherein the detectable label is an optically encoded fluorescent dye.

8. The composition of claim 7 formulated for flow cytometry analysis.

9. The composition of claim 1, wherein the individually addressable particle comprises a bead or a nanoparticle.

10. The composition of claim 1, wherein each individually addressable particle is separately labeled with a detectable label.

11. (canceled)

12. The composition of claim 1 formulated for research, industrial, medical, or veterinary use.

13-14. (canceled)

15. A kit comprising one or more compositions, each composition comprising individually addressable particles;
wherein each individually addressable particle comprises an external surface and having linked to said external surface a separate carbohydrate binding molecule; and
wherein each individually addressable particle is separately labeled with a detectable label.

16-21. (canceled)

22. A multiplex detection method for detecting a carbohydrate or a carbohydrate containing compound in a sample comprising:
contacting the sample with a solution comprising a plurality of individually addressable particles, each individually addressable particle comprising an external surface and having linked to said external surface a separate carbohydrate binding molecule; and
detecting the binding of the carbohydrate or carbohydrate containing compound to one more individually addressable particles;
wherein the carbohydrate or carbohydrate containing compound bound to one more individually addressable particles remains in suspension.

23. (canceled)

24. The method of claim 22, wherein each separate carbohydrate binding molecules is independently selected from the group consisting of lectins, antibodies, LECTENZ molecules (carbohydrate processing enzymes that have been inactivated but still bind to carbohydrate(s) with high specificity), carbohydrate-binding proteins, carbohydrate binding domains of proteins, pathogen adhesion domains (such as cholera toxin B, other toxins, and hemagglutinin), aptamers including protein, RNA or other small molecule aptamers, and any other molecule that naturally binds or is engineered to bind a carbohydrate.

25. The method of claim 24, wherein the individually addressable particle comprises a bead or a nanoparticle.

26. The method of claim 25, wherein each individually addressable particle is separately labeled with a detectable label.

27. The method of claim 26, wherein the detectable label is an optically encoded fluorescent dye.

28. The method of claim 27, wherein the detection is by flow cytometry analysis.

29. The method of claim 28, wherein at least one of the detected carbohydrates or carbohydrate containing compounds is detectable labeled.

30. The method of claim 29, co-detecting the detectably labeled individually addressable particle and the detectably labeled carbohydrates or carbohydrate containing compounds.

31-50. (canceled)
